(19) United States
(12) Patent Application Publication
(10) Pub. No.: US 2010/0034748 A1
(43) Pub. Date: Feb. 11, 2010

(54) Molecular imaging probes based on loaded reactive nano-scale latex

(76) Inventors: Guizhi Li, Rochester, NY (US); John William Harder, Rochester, NY (US); William James Harrison, Pittsford, NY (US)

Correspondence Address: Carestream Health, Inc., 150 Verona Street, Rochester, NY, 14608, US

(21) Appl. No.: 12/221,839

(22) Filed: Aug. 7, 2008

(51) Int. Cl. A61K 49/18; C08K 5/3417; B32B 1/00; A61K 49/04

(52) U.S. Cl. 424/9.3; 524/89; 524/90; 524/88; 428/402; 424/9.4

(57) ABSTRACT

The present invention relates to a loaded reactive nanoscale latex particle synthesized from mixture of monomers containing water insoluble monomers, at least two ethylenically functionalities monomers, halo-aromatic-polyethyleneglycol-methacrylate, polyethyleneglycolacrylate containing macromonomers, and up to 10 wt % other ethylenic monomers different from above monomers. The reactive halo-aromatic groups on the surface of latex particle are servable as linkers to react with peptides, antibodies, nucleic acids, ligands or other biomolecules.

### FIELD OF THE INVENTION

The present invention relates to a reactive nano-scale latex particle loaded with molecular imaging agents that is useful as molecular imaging probe. The reactive group on the latex particle surface easily reacts with peptides, antibodies, and other materials, for biological and diagnostic applications, particularly as fluorescent probes for Optical Molecular Imaging. More specifically, the invention relates to molecular imaging probes based loaded reactive nano-scale latex comprising a crosslinked polymer particle, wherein the crosslinked polymer is made from at least 45% water insoluble monomer and 1-30% monomers containing halo-aromatic reactive groups for reacting with peptides, ligands, nucleic acids, or proteins in aqueous dispersions. Yet more specifically, the present invention relates to a reactive nanolatex with a formula (X)m-(Y)n-(V)q-(T)o-(W)p wherein component (V) is halo-aromatic-polyethyleneglycol methacrylate.

### BACKGROUND OF THE INVENTION

Molecular imaging based techniques, especially optical molecular imaging, are very powerful tools for measuring the temporal and spatial dynamics of specific biomolecules and their interactions in vivo, protein function and gene expression in vivo. Optical imaging techniques have the great advantages over other techniques, such as magnetic resonance and X-ray. Optical imaging techniques have high resolution, high sensitivity, minimal invasion and can provide real-time operations. Nanoparticles provide the potential for simultaneous use of multiple probes and increased safety. These techniques have advanced over the past decade due to rapid developments in laser technology, sophisticated reconstruction algorithms and imaging software originally developed for non-optical, tomographic imaging modes such as computerized tomography (CT) and magnetic resonance imaging (MRI).

Nanoparticles have been increasingly used in a wide range of biomedical applications such as drug carriers and imaging agents. They are engineered materials with dimensions typically smaller than 100 nm, loaded with multiple molecules of contrast agents for multiple modalities imaging. Near-infrared (NIR) is defined as having a wavelength from 700 to 1000 nm. Near-infrared fluorescence (NIRF) imaging is of particular interest for non-invasive in vivo imaging because of the relatively low tissue absorbance, minimal autofluorescence of NIR light, and deep tissue penetration of up to 6-8 centimeters. A nanoparticle-based imaging probe has potential advantages over a small molecule or low molecular weight polymer-based probe, such as long blood circulating time.

In the past decades, much attention has been paid to fluorescent nanoparticles. Dyes have been incorporated into silica particles. (Ow, H.; Larson, D. R.; Srivastava, M.; Baird, B. A.; Webb, W. W.; Wiesner, U. “Bright and Stable Core-Shell Fluorescent Nanoparticles” Nano Letters 2005, 5, 113-117/Verhaegh, N. A. M.; Blaaderen, A. v. “Dispersions of Rhodamine-Labeled Silica Spheres: Synthesis, Characterization, and Fluorescence Confocal Scanning Laser Microscopy” Langmuir 1994, 10, 1427-1438. Imhof, A.; Megens, M.; Engelberts, J. J.; Lang, D. T. N. d.; Sprik, R.; Vos, W. L. “Spectroscopy of Fluorescein (FITC) Dyed Colloidal Silica Spheres”, J. Phys. Chem. B 1999, 103, 1408-1415.). Several reports have featured quantum dots (QDs) (Warren, C. W. et al., Science 1998, 281, 2016-2018) composed of a fluorescent core encapsulated within novel polymeric or lipid-based layers for NIRF optical imaging in cancer imaging in animals. However, most QDs are made of toxic material such as cadmium and it has not yet been established that QDs are non-toxic in the body.

WO2007120579 A2 relates to a loaded latex particle comprising a latex material made from a mixture represented by formula (X)m-(Y)n-(Z)o-(W)p, wherein Y is at least one monomer with at least two ethylenically unsaturated chemical functionalities; Z is at least one polyethylene glycol macromonomer with an average molecular weight of between 300 and 10,000; W is an ethylenic monomer different from X, Y, or Z; and X is at least one water insoluble, alkoxethyl containing monomer; and m, n, o, and p are the respective weight percentages of each monomer. The particle may be loaded with a fluorescent dye. However, the loaded latex particle doesn't contain a reactive halo-aromatic group on its surface.

Loaded latexes with IR dyes are known for inkjet and photographic applications (US 2002/0113854, U.S. Pat. No. 6,706,460). Latexes loaded with non-IR dyes are known for biological and diagnostic applications.

U.S. Pat. No. 7,033,524, issued Apr. 25, 2006, entitled “Polymer-based Nanocomposite Materials and Methods of Production Thereof” discloses the methods of producing polymer-based nanoparticles via emulsion polymerization techniques to generate composite materials. The core materials include polymer or inorganic based oxide and the core was coated with a layer of polymer as a shell. However, there is no chemical bonding between cores and shells.

U.S. Pat. No. 6,964,844 relates generally to the synthesis of novel dyes and labels and methods for the detection or visualization of analytes and more specifically to fluorescent latex particles which incorporate the novel fluorescent dyes and utilize, in certain aspects, fluorescence energy transfer and intramolecular energy transfer, for the detection of analytes in immunoassays or in nucleic acid assays. These dyes are water soluble hybrid phthalocyanine derivatives useful in competitive and noncompetitive assays immunoassays, nucleic acid and assays are disclosed and claimed having (1) at least one donor subunit with a desired excitation peak; and (2) at least one acceptor subunit with a desired emission peak, wherein said derivative(s) is/are capable of intramolecular energy transfer from said donor subunit to said acceptor subunit. Such derivatives also may contain an electron transfer subunit. Axial ligands may be covalently bound to the metals contained in the water soluble hybrid phthalocyanine derivatives. Ligands, ligand analogues, polypeptides, proteins and nucleic acids can be linked to the axial ligands of the dyes to form dye conjugates useful in immunoassays and nucleic acid assays.

U.S. Pat. No. 4,997,772 relates to a core/shell polymer particle containing a detectable tracer material in the core only. It also relates to an immunoreactive reagent and the use of that reagent in analytical elements and methods. A water-insoluble polymeric particle has an inner core comprising a detectable tracer material distributed in a first polymer for which the tracer material has a high affinity. This first polymer has a glass transition temperature (Tg₁) less than about 100 degree C. The particle also has an outer shell comprising a second polymer for which the tracer material has substantially less affinity relative to said first polymer. This second polymer has a glass transition temperature (Tg₂), which is greater than or equal to the term [Tg₁−10 degree C.]. It also contains groups which are either reactive with free amino or sulfhydryl groups of an immunoreactive species or which can be activated for reaction with such groups. Such a species can be covalently attached to this particle to form an immunoreactive reagent which is useful in analytical elements and various analytical methods including immunological methods, for example, agglutination assays. However, the particles contain two polymers with different glass transition temperatures, one formed the core and another formed the shell.

U.S. 2004/0038318 relates to a reagent set, as well as to a method, for carrying out simultaneous analyses of multiple isoenzymes in a test sample, particularly a bodily fluid. The method is useful for measuring creatine kinase isoenzymes in particle, or bead, based multiplexed assay systems.

U.S. Pat. No. 4,891,324 relates to methods for performing an assay for determining an analyte by use of a conjugate of a member of a specific binding pair consisting of ligands and receptors, with a particle. The method has particular application to heterogeneous immunoassays of biological fluids, for example, serum or urine. The method is carried out using a composition that includes a conjugate of a first specific binding pair member with a particle. A luminescer is reversibly associated with a nonaqueous phase of the particle. Where the first specific binding pair member is not complementary to the analyte, a second specific binding pair member that is capable of binding to the first specific binding pair member is employed. Unbound conjugate is separated from conjugate that is bound to the analyte or to the second specific binding pair member. A reagent for enhancing the detectability of the luminescer is added and the light emission of the luminescer acted on by the reagent is measured.

WO 2006/016166 relates to polymeric materials suitable for medical materials. This invention discloses a polymer containing an alkoxyethyl acrylate monomer, a monomer containing a primary, secondary, tertiary or quaternary amine group and a monomer containing an acid group. The polymer composition forms fibers with the preferred size of 0.5 to 2.0 um, which is still not sufficient to provide nanoparticles less than 100 nm in size which are colloidally stable and can be loaded with non-water soluble fluorescent dye for the purposes of diagnostic imaging.

U.S. Pat. No. 5,326,692 relates to polymeric materials incorporating multiple fluorescent dyes to allow for controlled enhancement of the Stokes shift. In particular, the invention describes microparticles incorporating a series of two or more fluorescent compounds having overlapping excitation and emission spectra, resulting in fluorescent microparticles with a desired effective Stokes shift. The novel fluorescent microparticles are useful in applications such as the detection and analysis of biomolecules, such as DNA and RNA, that require a very high sensitivity and in flow cytometric and microscopy analytical techniques. The invention relates to microparticles incorporating a series of two or more fluorescent dyes having overlapping excitation and emission spectra allowing efficient energy transfer from the excitation wavelength of the first dye in the series, transfer through the dyes in the series and re-emitted as an optical signal at the emission wavelength of last dye in the series, resulting in a desired effective Stokes shift which is controlled through selection of appropriate dyes.

In general, IR-emissive nano-assemblies for physiological imaging have several problems.

First, the dyes are often highly aggregated and hence fluorescence quenched.

Second, the fluorescence for the dye-nanoparticle assemblies is often inefficient in an aqueous environment. The dye requires a high salt content to remain in solution in a biological fluid.

Third, the dyes used in IR-emissive nano-assemblies are unstable to light, oxygen water, and bleach readily. IR dyes are especially susceptible to environmental conditions causing the dye to lose the ability to absorb and emit light.

Fourth, IR-emissive nano-assemblies are often colloidally unstable and cytotoxic.

Fifth, some dye-nanoparticle assemblies are difficult to react directly with peptides or ligands in aqueous solution.

Therefore, there exist there is a need a latex loaded nanoscale particle having a halo-aromatic-polyethyleneglycol methacrylate.

### SUMMARY OF THE INVENTION

An object of the present invention is to provide a nano-scale latex containing a dye having good fluorescence properties.

Another object of the present invention is to provide latex particle that is soluble in an aqueous environment.

A further object of the present invention is to provide a dye that is stable to light, oxygen, water and has good fade resistance.

A yet further object of the present invention is to provide a latex particle that is colloidally stable and non-cytotoxic.

Another object of the present invention is to provide a latex particle that is capable of reacting directly with peptides and ligands in aqueous solution.

These objects are given only by way of illustrative example, and such objects may be exemplary of one or more embodiments of the invention. Other desirable objectives and advantages inherently achieved by the disclosed invention may occur or become apparent to those skilled in the art. The invention is defined by the appended claims.

According to one aspect of the invention, there is provided a molecular imaging probe based loaded reactive nano-scale latex comprising a crosslinked polymer particle, wherein the crosslinked polymer is synthesized from a mixture of monomers containing water insoluble monomers, at least two ethylenically functionalities monomers, halo-aromatic-polyethyleneglycol-methacrylate, polyethyleneglycol acrylate containing macromonomers, and up to 10 wt % other ethylenic monomers different from above monomers. The nano-scale latex is loaded with a molecular imaging agent.

### BRIEF DESCRIPTION OF THE DRAWINGS

The foregoing and other objects, features, and advantages of the invention will be apparent from the following more particular description of the embodiments of the invention, as illustrated in the accompanying drawings.

FIG. 1 shows a graph of the UV-visible absorption spectra of reactive nanolatex (20 wt % 4-fluoro-2-nitro-benzoyl-PEG-MA) conjugated with lysine for 0 h, 2 h, 7 h and 24 h.

FIG. 2 shows a graph of the UV-visible absorption spectra of reactive nanolatex LRL-4A (5 wt % 4-fluoro-2-nitro-benzoyl-PEG-MA) conjugated with lysine for 0 h, 2 h, 7 h and 24 h.

FIG. 3 shows a graph of the UV-visible absorption spectra of reactive nanolatex LRL-5A (15 wt % 4-fluoro-2-nitro-benzoyl-PEG-MA) conjugated with IgG antibody for 0 h, 5 and 24 h.

FIG. 4 shows a graph of the UV-visible absorption spectra of reactive nanolatex 6 (10 wt % 4-fluoro-3-nitro-benzoyl-PEG-MA) conjugated with lysine for 0 h, 2 h, 7 h, 24 h and 31 h.

FIG. 5 shows a halo-aromatic polyethylene glycol co-polymer reacted with a peptide in water.

### DETAILED DESCRIPTION OF THE INVENTION

The following is a detailed description of the preferred embodiments.

The loaded reactive nanolatex particles combine several advantageous properties that make them well suitable for specific biological and diagnostic applications. In addition to providing good fluorescence efficiencies, they are highly biocompatible, are resistant to adhesion of serum proteins, and remain well dispersed in aqueous solution or saline buffer over a couple of months. Furthermore, the reactive functional groups, such as fluoro-nitro-benzoyl on the reactive nanolatex surface, can react directly with bioactive compounds, peptides, ligands, and proteins, in aqueous dispersions under mild conditions.

The loaded reactive nanolatex particles are molecular imaging agents, especially useful are hydrophobic visible and hydrophobic infrared dyes, non-covalently loaded into heavily PEGylated nanolatex particles with reactive groups on surface, when preferably used in near IR-active assemblies show highly efficient fluorescence, low dye aggregation, and high photostability, that is, they are less subject to bleaching. The assemblies are also non-cytotoxic and are very colloidally stable, that is, are less prone to form aggregation. In one embodiment, the reactive nanolatex particle is a crosslinked polymer, has a hydrodynamic diameter less than 100 nm, is composed of alkoxyethyl methacrylate or alkoxyethyl acrylate monomers and is further composed of at least one poly(ethylene glycol)-methacrylate (PEG-MA) with halo-aromatic reactive groups at the end of PEG macromonomers. In one embodiment, the halo-aromatic reactive group is fluoro-nitro-benzoyl.

“PEGylated” refers to nanolatex compositions which are composed of at least 20 weight percent covalently bound poly(ethylene glycol).

“Pegylation” typically refers to the reaction by which a PEG-protein/peptide/ligand conjugate is formed. This also applies to PEG-therapeutic agent, PEG-dye, PEG-bioactive ligand, PEG-(MRI contrast agent), PEG-(X-Ray contrast agent), PEG-(positron emission tomography (PET) compounds), PEG-peptides(cell penetrating TAT peptide, tumor-targeting peptides, e.g. anti-HER2 neu peptide (AHNP-dF, AHNP-Y)), PEG-antibody (or antibody fragments), PEG-(enzyme inhibitor), PEG-(radioactive isotope), PEG-(quantum dot), PEG-oligosaccharide, PEG-polygosaccharide, PEG-hormone, PEG-dextran, PEG-oligonucleotide, PEG-carbohydrate, PEG-neurotransmitter, PEG-hapten, PEG-carotinoid, and PEG-drug.

“Nanolatex” refers to a hydrophobic polymeric particle, which has a hydrodynamic diameter of less than 100 nm.

A “hydrophobic crosslinked polymer” refers to a polymer made of at least 45 weight percent of water-insoluble monomers. The polymer is a contiguous network through which a through-bond path can be traced between any two atoms (not including counter ions).

A “water dispersible crosslinked polymeric particle” refers to a polymer particle which is a contiguous, crosslinked polymer network through which a through-bond path can be traced between any two atoms (not including counter ions) in the particle. The particle can exist in water in such status that each individual network particle is separated from every other by aqueous continuous phase.

“Biocompatible” means that a composition does not disrupt the normal function of the bio-system into which it is introduced. Typically, a biocompatible composition will be compatible with blood and does not otherwise cause an adverse reaction in the body. For example, to be biocompatible, the material should not be toxic, immunogenic or thrombogenic.

“Colloidally stable” refers to the state in which the particle is capable of existing in aqueous phosphate buffered saline. For example, 137 mM NₐCl, 2.7 mM KCl, 10 mM Na₂HPO₄, 2 mM KH₂PO₄ at pH 7.4. In such dispersion state each individual nanolatex particle is separated from every other by the aqueous continuous phase without the formation of agglomerates or without bulk flocculation occurring.

“Loaded” refers to a non-covalent interaction between the loaded agents (dyes, other imaging agents, or drug, or other agents) and the component of polymer particle such that when the nanolatex is dispersed in water at a concentration of less than 10%, less than 1% of the total dye in the system can be extracted into the water continuous phase.

“Labeling” refers to the attachment of the loaded reactive latex or loaded latex conjugate to a material to aid in the identification of the material. Preferably, the material is identified by optical detection.

“Biodegradable” means that the material can be degraded either enzymatically or hydrolytically under physiological conditions to smaller molecules that can be eliminated or excreted from the body through normal processes.

The term “diagnostic agent” includes components that act as contrast agents and thereby produce a detectable indicating signal in the host or test sample. The detectable signal includes, but is not limited to, gamma-emitting, radioactive, echogenic, fluoroscopic or physiological signals.

The term “biomedical agent” as used herein includes biologically active substances which are effective in the treatment of a physiological disorder, pharmaceuticals, antibodies, enzymes, hormones, steroids, recombinant products.

The nanolatex is composed of repetitive crosslinked ethylenically unsaturated monomers. The nanolatex has a volume-average hydrodynamic diameter from 5 nm to 100 nm, preferably 8 to 60 nm as determined by quasi-elastic light scattering in phosphate buffered saline (137 mM NaCl, 2.7 mM KCl, 10 mM Na₂HPO₄, 2 mM KH₂PO₄ at pH 7.4.).

In one embodiment, the loaded reactive latex particle contains a latex made from a mixture of monomers presented by the following Formula 1:

(X)m-(Y)n-(V)q-(T)o-(W)p Formula 1

X is at least one water insoluble, alkoxethyl containing monomer; Y is at least one monomer with at least two ethylenically unsaturated chemical functionalities; V is halo-aromatic functionalized poly(ethylene glycol) methacrylate with an average molecular weight from 300 to 6,000; T is at least one nonfunctional or functional polyethylene glycol macromonomer with an average molecular weight of between 300 and 10,000; and W is an ethylenic monomer different from X, Y, V or T. The weight percent range of each component monomer is represented by m, n, q, o, and p: m ranges between 40-80 wt %, preferably from 45-60 wt %; n ranges between 1-10 wt %, preferably 2-6 wt %; q ranges between 1-30 wt %, preferably 5-20wt %; o ranges between 10-60 wt %, preferably between 20-50 wt %, and p is up to 10 wt %.

In Formula 1, X is a water-insoluble, alkoxyethyl-containing monomer as shown in Formula 2:

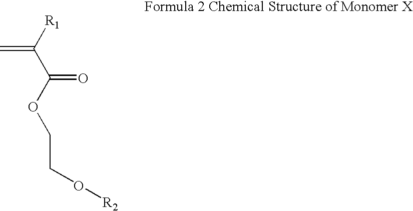

R1 is methyl or hydrogen. R2 is an alkyl or aryl group containing up to 10 carbons. In one embodiment, X is methoxyethyl methacrylate or alkoxyethyl acrylate.

Referring again to Formula 1, Y is a water-insoluble or water-soluble monomer containing at least two ethylenically unsaturated chemical functionalities. These functionalities include vinyl groups, acrylates, methacrylates, acrylamides, methacrylamides, allyl groups, vinyl ethers and vinyl esters. Y monomers include, but are not necessarily limited to aromatic divinyl compounds such as divinylbenzene, divinylnaphthalene or derivatives thereof, diethylene carboxylate esters and amides, such as ethylene glycol dimethacrylate, poly(ethylene glycol)-dimethacrylate, 1,4 butanediol diacrylate, 1,4 butanediol dimethacrylate, 1,3 butylene glycol diacrylate, 1,3 butylene glycol dimethacrylate, cyclohexane dimethanol diacrylate, cyclohexane dimethanol dimethacrylate, diethylene glycol diacrylate, diethylene glycol dimethacrylate, dipropylene glycol diacrylate, dipropylene glycol dimethacrylate, ethylene glycol diacrylate, 1,6 hexanediol diacrylate, 1,6 hexanediol dimethacrylate, neopentyl glycol diacrylate, neopentyl glycol dimethacrylate, tetraethylene glycol diacrylate, tetraethylene glycol dimethacrylate, triethylene glycol diacrylate, triethylene glycol dimethacrylate, tripropylene glycol diacrylate, tripropylene glycol dimethacrylate, pentaerythritol triacrylate, trimethylolpropane triacrylate, trimethylolpropane trimethacrylate, dipentaerythritol pentaacrylate, di-trimethylolpropane tetraacrylate, pentaerythritol tetraacrylate, divinyl esters such as divinyl adipate, and other divinyl compounds such as divinyl sulfide or divinyl sulfone compounds of allyl methacrylate, allyl acrylate, cyclohexanedimethanol divinyl ether diallylphthalate, diallyl maleate, dienes such as butadiene and isoprene and mixtures thereof.

Monomer “V” as defined in Formula 1, is a reactive polyethyleneglycol methacrylate derivative of Formula 3A or 3B:

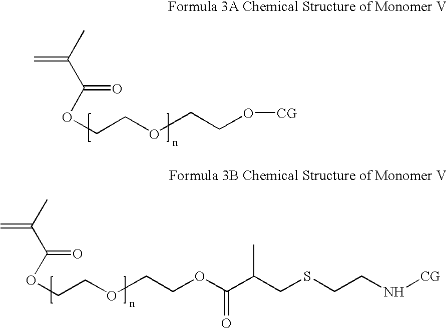

wherein n is greater than 1 and less than 200, preferably 5 to 110. CG is 4-halo-3-nitrobenzoyl, 2-halo-3-nitrobenzoyl, 2-halo-4-nitrobenzoyl, 4-halo-2-nitrobenzoyl, 2-halo-5-nitrobenzoyl, 3-halo-2-nitrobenzoyl, 2-halonicotinate, 4-halonicotinate, 6-halonicotinate, 2-haloisonicotinate, or 3-haloisonicotinate; where halo is fluoro, chloro, bromo, or iodo.

In one embodiment, CG is selected from the group of structures:

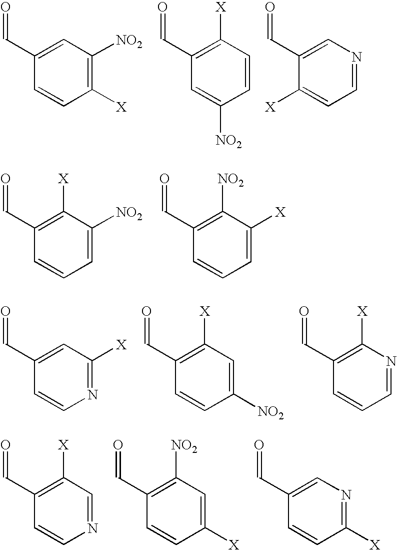

Where X is a halo selected from fluoro, chloro, bromo, and iodo.

The macromonomers have at least two reactive groups: The first reactive group being an acrylate useful for forming nanolatex crosslinked network. The second being a halo-aromatic reactive group at the end of PEG, as shown in Formula 3, which is useful for direct reacting with targeting compounds or ligands, contrast agents, dyes, proteins, amino acids, peptides, antibodies, antibody fragments, bioactive ligands, phages, phage fragments, therapeutic agents, metal chelating agents, nucleic acid molecules, oligonucleotides and enzyme inhibitors.

The reactive halo-aromatic groups on the nanolatex surface are useful for attachment to biologically important materials, targeting peptides, ligands, proteins, antibodies, cells, dyes, drugs, contrast agents, therapeutic agents and thickener agents. Contrast agents are used for detection and diagnostics of disease and the study of metabolic activity, in methods such as PET, MRI, single photon emission computerized tomography (SPECT)/CT. Therapeutic agents are used for the treatment of disease. Thickener agents are useful for making pharmaceuticals, and cosmetics. The preferred biologically important materials for the attachment include targeting agents such as, targeting peptides, proteins, ligands, targeting antibody and fragments; diagnostic agents; and therapeutic agents, which can be greatly improved in effectiveness when linked by attachment.

The reactive halo-aromatic functionality allows the loaded reactive nanolatex particles to be covalently bonded to a biomolecule, bioactive ligands, or cells and the location of the biomolecule, bioactive ligands and cells can be determined by fluorescent imaging or other molecular imaging techniques such as PET, MRI, or CT. The covalent attachment provides a link that is stable to handling, changes in solvent, pH, ionic strength, and temperature. This stable covalent bond between the loaded nanolatex particle and the biomolecule is important to assure that the fluorescent signal that is detectable and relates to the presence of the biomolecule. The halo-aromatic reactive functional group on the nanolatex surface is easily reacted with primary or secondary amines, which are from amino acids, peptides, polypeptides, targeting agents, proteins, antibodies, antibody fragments, bioactive compounds, ligands, phages, phage fragments, diagnostic agents, molecular imaging probes, cells, RNA, DNA, RNA and DNA sequences, drugs or pharmaceutical/biomedical agents.

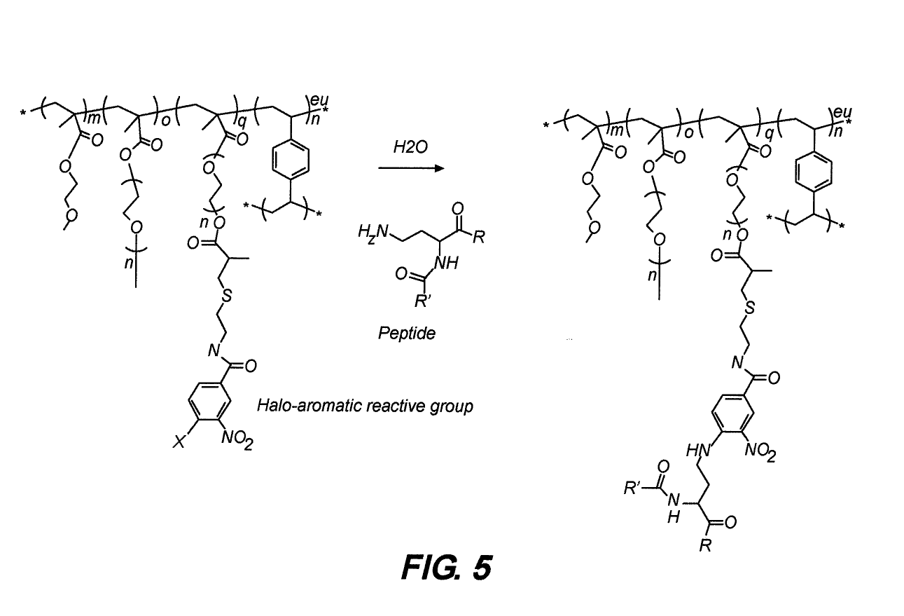

As shown in FIG. 5 the halo-aromatic polyethylene glycol acrylate co-polymer is reacted with a peptide in water. The leaving group X is displaced from the halo-aromatic polyethylene glycol acrylate co-polymer and the peptide is covalently attached. The reactivity of the leaving groups enables the reaction to proceed in water at mild conditions to protect bioactive molecules that are sensitive to acid, base, high temperatures, ionic strength, and organic solvents. The leaving group X is chosen such that the aromatic reactive group has sufficient stability in water without decomposing but facile reaction with bio-active molecules such that covalent bonding can occur under mild conditions. As discussed above in one embodiment, halogen leaving groups are utilized. The comparative reactivity of the halo groups is fluoro >chloro>bromo>iodo. However it is understood that other leaving groups can be used such as aromatic sulfonates, alky sulfonates, electron withdrawing phenols and hetercyclic thiols. In one embodiment, the leaving group is toluene sulfonate, methane sulfonates, trfluoromethane sufonate, 2,4-dinitrophenol or mercaptotetrazoles

Referring again to Formula 1, the W monomer consists of any other inert monomers which are added to modify the desired properties. W is a non-chemically reactive monomer which is added in small amounts to impart desirable properties to the latex. Desirable proprieties include, but are not limited to water dispersibility, charge, more facile dye loading, or to make the latex more hydrophobic. W may be a water-soluble monomer such as 2-phosphatoethyl acrylate potassium salt, 3-phosphatopropyl methacrylate ammonium salt, vinylphosphonic acid, and their salts, vinylcarbazole, vinylimidazole, vinylpyrrolidone, vinylpyridines, acrylaminde, methacrylamide, maleic acid and salts thereof, sulfopropyl acrylate and methacrylate, acrylic and methacrylic acids and salts thereof, N-vinylpyrrolidone, acrylic and methacrylic esters of alkylphosphonates, styrenics, acrylic and methacrylic monomers containing amine or ammonium functionalities, styrenesulfonic acid and salts thereof, acrylic and methacrylic esters of alkylsulfonates, vinylsulfonic acid and salts thereof, vinylpyridines, hydroxyethyl acrylate, glycerol acrylate and methacrylate esters, (meth)acrylamide, and N-vinylpyrrolidone. W may alternately be a water-insoluble monomer such as methyl methacrylate, ethyl methacrylate, isobutyl methacrylate, 2-ethylhexyl methacrylate, benzyl methacrylate, cyclohexyl methacrylate and glycidyl methacrylate, acrylic/acrylate esters such as methyl acrylate, ethyl acrylate, isobutyl acrylate, 2-ethylhexyl acrylate, benzyl methacrylate, phenoxyethyl acrylate, cyclohexyl acrylate, and glycidyl acrylate, styrenics such as styrene, a-methylstyrene, ethylstyrene, 3- and 4-chloromethylstyrene, halogen-substituted styrenes, and alkyl-substituted styrenes, vinyl halides and vinylidene halides, N-alkylated acrylamides and methacrylamides, vinyl esters such as vinyl acetate and vinyl benzoyl, vinyl ether, allyl alcohol and its ethers and esters, and unsaturated ketones and aldehydes such as acrolein and methyl vinyl ketone, isoprene, butadiene and acrylonitrile.

T is a water-soluble polyethylene glycol macromonomer with a molecular weight of between 300 and 10,000, preferably between 500 and 5000. In one embodiment, the linking polymer is a polyethylene glycol backbone chain with nonfunctional methoxyl-, or hydroxyl, or specific functional end groups at each end, which allows the polyethylene glycol to act as a linking group. The polyethylene glycol macromonomer contains a radical polymerizable group at the other end. This group can be, but is not necessarily limited to a methacrylate, acrylate, acrylamide, methacrylamide, styrenic, allyl, vinyl, maleimide, or maleate ester. This functional group may be, but is not limited to hydroxyls, carboxylic acids, vinylsulfonyls, aldehydes, epoxides, succinimidyl esters and maleimides. In a preferred embodiment, these functional groups are hydroxyl, carboxylic acids or maleimides.

A preferred class of polyethylene glycol macromonomers, defined as T in Formula 1, is described by Formula 4:

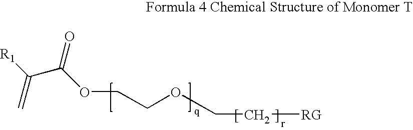

In Formula 4, R1 is hydrogen or methyl, q is 5-220, r is 1-10, and RG is hydrogen, or functional group selected from hydroxyl, carboxylic acid, vinylsulfonyl, aldehyde, epoxides, succinimidyl ester, maleimide, a substituted or unsubstituted acetate, or substituted carbamyl, substituted phosphate, substituted or unsubstituted sulfonate. The functional group should allow covalent bonding reaction to occur in organic solvents such as N,N-dimethylformamide, tetrahydrofuran, dimethylsulfoxide, N-methylpyrrolidone, non-organic solvents such as water, or their mixtures.

By the proper use of reactive fluoro-nitro-benzoyl groups or other functional groups, the loaded reactive nanolatex can be covalently attached to any drug or biomolecule in such a way to optimize the fluorescent signal and not interfere with the normal function of the biomolecule and avoid aggregation of reactive nanoparticles or aggregation of nanoparticle-biomolecule conjugates. For instance, if primary or secondary amines were used as the RG group in monomer T, there is a potential for reaction with the fluoro-nitro-benzoyl functionality on monomer V, undesirably resulting in the aggregation of particles. Fluoro-nitro-benzoyl functionality allows the latex to react directly with biomolecules, ligands, peptides, antibodies and fragments, or proteins in aqueous solution at 37° C. (i.e. normal body temperature) without the need for additional linker groups.

A carboxylic acid attachment group can be converted to an active ester to enable formation of a covalent bond. In one embodiment, N-hydroxysuccinimide ester is used in the activating the carboxylic acid group. In another embodiment the carboxylic acid attachment group is activated for covalent bond formation by carbodiimide reageants, such as dicylcohexylcarbodiimide (DCC), or 1-ethyl-3-(3-dimethylaminopropyl) carbodiimide hydrochloride (EDAC).

A hydroxyl attachment group can be activated for covalent bond formation by forming a chloroformate such as p-nitrophenyl chloroformate.

The maleimide linking groups are capable of reacting with thiol groups typically available from cysteine residues in biomolecules or a thiol linking group from the list above. Trialkoxysilane is useful for reacting with other trialkoxysilanes or siloxide modified molecules or particles. Alkyne and azidoyl groups are useful for forming a stable triazole link often catalyzed by copper (I); such that if the dye contains an alkynyl attachment group, then an azidoyl attachment group is placed on the biomolecule. Alternatively, the azidoyl group is the attachment group on the dye and an alkynyl group is added to the biomolecule.

Targeting agents are compounds, peptides, ligands, nucleic acids, antibodies or their fragments or proteins, with specific groups that will identify and associate with a specific site, such as a disease site, such that the particle or conjugated material will be concentrated in the site for enhanced effect. Also of particular interest are nanolatex-antibody/peptides/ligands conjugates. Antibodies, also known as immunoglobulins (Ig's), are proteins that help identify foreign substances to the immune system, such as a bacteria or a virus or any substance containing an antigen, and are useful for identification and association of specific biological targets. Bioactive ligands and peptides have specific useful groups that are associated with, and bind to receptors expressed in or on cells or with enzymes, or in disease area. Examples of bioactive ligands are growth factors, such as biotin and folic acid, RGD tumor targeting peptide, anti-HER2/neu peptide (AHNP), TAT cell penetrating peptide, specific proteins and peptide, sequences of amino acids or molecules, which have strong binding ability or affinity to the active sites of enzymes, specific cells and disease sites, or help the nanolatex particles to penetrate or concentrate on or in cells of interest.

Diagnostic agents are compounds or materials which enhance the signal of detection when a material is scanned with light, sound, magnetic, electronic and radioactive sources of energy. Examples are dyes, such as UV, visible or infrared absorbing dyes, fluorescent dyes, including indocarbocyanines and fluorescein; MRI contrast agents, such as gadallinium, and iron oxide complexes or compounds or nanoparticles; X-ray contrast agents, such as a polyiodoaromatic compound; and positive emission tomography (PET) agents, such as ¹¹C, ¹⁸F, ⁶⁴Cu compounds or other positron emitter chemicals. In one embodiment the loaded nanolatex particles is functionalized with chelating groups carrying a radioisotope or metal used for MR. Components having a short half life can be mixed immediately prior to injection to bond with the chelating group. Suitable chelating groups include diethylenteriamepenatacetic acid (DTPA) or 1,4,7,10-tetra-azacyclododecane-N,N′,N″,N′″-tetra acetic acid (DOTA). These chelating groups allow the chelating of metals, such as Gadolinium used in MRI and X-ray imaging. Tetra- and pentaacetic acid chelating groups further allow the loaded nanolatex to be labeled with radioisotopes for radioscintigraphy, single photon emission computerized tomography (SPECT) and PET.

The component being labeled can be in a mixture including other materials. In one embodiment the mixture, in which the labeling reaction occurs, is a liquid mixture, particularly a water mixture. The detection step is performed with the mixture in a liquid or dry condition, such as a microscope slide.

The component or conjugate to which the loaded latex is attached, also referred to as the labeled component, can be antibodies, antibody fragments, proteins, peptides, polypeptides, phages, phage fragments, enzyme substrates, hormones, lymphokines, metabolites, receptors, antigens, haptens, lectins, toxins, carbohydrates, sugars, oligosaccharides, polysaccharides, nucleic acids, deoxy nucleic acids, derivatized nucleic acids, derivatized deoxy nucleic acids, DNA sequences, RNA sequences, derivatized DNA sequences, derivatized RNA sequences, natural drugs, virus particles, bacterial particles, virus components, yeast components, blood cells, blood cell components, biological cells, noncellular blood components, bacteria, bacterial components, natural and synthetic lipid vesicles, synthetic drugs and medicines, poisons, environmental pollutants, polymers, polymer particles, glass particles, glass surfaces, plastic particles and plastic surfaces.

A variety of loaded latex-conjugates may be prepared by using the loaded reactive latexes of the invention to conjugate with antigens, antibodies, antibody fragments, phages, phage fragments, steroids, vitamins, drugs, haptens, metabolites, toxins, environmental pollutants, amino acids, peptides, proteins, nucleic acids, nucleic acid sequences, carbohydrates, lipids, and polymers. In another embodiment, the conjugated substance is an amino acid, peptide, protein, polysaccharide, nucleotide, oligonucleotide, nucleic acid, hapten, drug, lipid, phospholipid, lipoprotein, lipopolysaccharide, liposome, lipophilic polymer, polymer, polymeric microparticle, biological cell or virus. In one aspect of the invention, the conjugated substance is labeled with a plurality of loaded latexes of the present invention, which may be the same or different.

The loaded reactive latexes and loaded latex-conjugate are useful as labels for molecular imaging probes in immunoassays and also as labels for in-vivo imaging and in-vivo tumor therapy or other disease therapy.

In one embodiment, the loaded reactive latexes are used as agents for in-vivo imaging. When used as imaging agents, these loaded latexes are conjugated to one member of a specific binding pair to give a labeled conjugate/binding complement. The loaded latex-conjugate is introduced into an animal. If the other member of the specific binding pair is present, the loaded latex-conjugate will bind thereto and the signal produced by the dye is capable of being measured and its localization identified.

The loaded latexes are also useful for in-vivo tumor therapy. For example, photodynamic therapy involves using an additional dye component attached to the surface of the nanoparticle as a photosensitizing agent. The loaded latex with photosensitizing agent is further conjugated to a binding bioactive ligands or peptides (RGD cyclic peptide, folic acid, or AHNP targeting peptide) which may specifically recognize and bind to a component (folate, or HER2 receptors for the targeting peptides) of a tumor cell. The localized triplet emission from the bound dye-loaded latex conjugate after excitation by light, causes chemical reactions and selective damage and/or destruction to the tumor cells.

Target Analyte: In one embodiment, the loaded reactive latex or loaded latex-conjugates are used to probe a sample solution for the presence or absence of a target analyte. By “target analyte” or “analyte” or grammatical equivalents herein is meant any atom, molecule, ion, molecular ion, compound, ligands, particle, or cell to be either detected or evaluated for binding partners. As will be appreciated by those in the art, a large number of analytes may be used in the present invention; basically, any target analyte can be used which binds a bioactive agent or for which a binding partner (i.e. drug candidate) is sought. The target material is optionally a material of biological or synthetic origin that is present as a molecule or as a group of molecules, including, but not limited to, antibodies, antibody fragments, phages, phage fragments, amino acids, proteins, peptides, polypeptides, enzymes, enzyme substrates, hormones, lymphokines, metabolites, antigens, haptens, lectins, avidin, streptavidin, toxins, poisons, environmental pollutants, carbohydrates, oligosaccarides, polysaccharides, glycoproteins, glycolipids, nucleotides, oligonucleotides, nucleic acids and derivatized nucleic acids (including deoxyribo-and ribonucleic acids), DNA and RNA sequences and derivatized sequences (including single and multi-stranded sequences), natural and synthetic drugs, receptors, virus, virus particles, bacterial particles, virus components or fragments, biological cells, spores, cellular components (including cellular membranes and organelles), natural and synthetic lipid vesicles, polymer membranes, polymer surfaces and particles, and glass and plastic surfaces and particles. Typically the target material is present as a component or contaminant of a sample taken from a biological or environmental system. Particularly preferred analytes are nucleic acids, targeting peptides, targeting ligands, antibodies, and proteins.

In one embodiment, the conjugate is a bioreactive substance. The target material is optionally a bioreactive substance. Bioreactive substances are substances that react with or bind to molecules that are derived from a biological system, whether such molecules are naturally occurring or result from some external disturbance of the system (e.g. disease, poisoning, genetic manipulation). By way of illustration, bioreactive substances include biomolecules (i.e. molecules of biological origin including, without limitation, polymeric biomolecules such as pepfides, polypeptides, proteins, polysaccharides, oligonucleotides, avidin, streptavidin, neutravidin, phage and phage fragments, DNA and RNA, as well as non-polymeric biomolecules such as biotin and digoxigenin and other haptens typically having a MW less than 1000), microscopic organisms such as viruses and bacteria, and synthetic haptens (such as hormones, vitamins, or drugs). Typically the target complement or the target material or both are amino acids, peptides (including polypeptides), or proteins (larger MW than polypeptides); or are nucleotides, oligonucleotides (less than 20 bases), or nucleic acids (i.e. polynucleotides larger than oligonucleotides, including RNA and single-and multi-stranded DNA and sequences and derivatized sequences thereof); or are carbohydrates or carbohydrate derivatives, including monosaccharides, polysaccharides, oligosaccharides, glycolipids, and glycoproteins; or are haptens (a chemical compound that is unable to elicit an immunological response unless conjugated to a larger carrier molecule), which haptens are optionally conjugated to other biomolecules; or a microscopic organisms or components of microscopic organisms. For such bioreactive substances, there are a variety of known methods for selecting useful pairs of corresponding conjugates complementary to the target materials.

Where more than one material is targeted simultaneously, multiple conjugates which are target complements (one for each corresponding target material) are optionally included. Target complements are selected to have the desired degree of specificity or selectivity for the intended target materials or diseases. The nanoscale latex particle is capable of carrying a plurality of targeting molecules.

In one embodiment, the target analyte is a protein. As will be appreciated by those in the art, there are a large number of possible proteinaceous target analytes that may be detected or evaluated for binding partners using the present invention. Suitable protein target analytes include, but are not limited to, (1) immunoglobulins; (2) enzymes (and other proteins); (3) hormones and cytokines (many of which serve as ligands for cellular receptors); and (4) other proteins. In a preferred embodiment, the target analyte is a nucleic acid. In a preferred embodiment, the probes are used in genetic diagnosis. For example, probes can be made using the techniques disclosed herein to detect target sequences such as the gene for nonpolyposis colon cancer, the BRCA1 breast cancer gene, P53, which is a gene associated with a variety of cancers, the Apo E4 gene that indicates a greater risk of Alzheimer's disease, allowing for easy presymptomatic screening of patients, mutations in the cystic fibrosis gene, or any of the others well known in the art.

In an additional embodiment, viral and bacterial detection is done using the complexes of the invention. In this embodiment, probes are designed to detect target sequences from a variety of bacteria and viruses. For example, current blood-screening techniques rely on the detection of anti-HIV antibodies. The methods disclosed herein allow for direct screening of clinical samples to detect HIV nucleic acid sequences, particularly highly conserved HIV sequences. In addition, this allows direct monitoring of circulating virus within a patient as an improved method of assessing the efficacy of anti-viral therapies. Similarly viruses associated with leukemia, HTLV-I and HTLV-II, may be labeled and detected in this way. Bacterial infections, such as tuberculosis, clymidia and other sexually transmitted diseases, may also be detected by using labeled virus, phage or fragments.

In another embodiment, the nucleic acids or nucleic acid-loaded latex conjugates of the invention find use as probes for toxic bacteria in the screening of water and food samples. For example, samples may be treated to lyse the bacteria to release its nucleic acid, and then probes designed to recognize bacterial strains, including, but not limited to, such pathogenic strains as, Salmonella, Campylobacter, Vibrio cholerae, Leishmania, enterotoxic strains of E. coli, and Legionnaire's disease bacteria.

The described composition can further comprise a biological, pharmaceutical or diagnostic component that includes a targeting moiety that recognizes a specific target cell. Recognition and binding of a cell surface receptor through a targeting moiety associated with loaded latex nanoparticle can be a feature of the described compositions. This feature takes advantage of the understanding that a cell surface binding event is often the initiating step in a cellular cascade leading to a range of events, notably receptor-mediated endocytosis. The term “receptor mediated endocytosis” generally describes a mechanism by which, activated by the binding of a ligand to a receptor displayed on the surface of a cell, a receptor-bound ligand is internalized within a cell. Many proteins and other structures enter cells via receptor mediated endocytosis, including insulin, epidermal growth factor, growth hormone, thyroid stimulating hormone, nerve growth factor, calcitonin, glucagon and many others.

Receptor Mediated Endocytosis (RME) affords a convenient mechanism for transporting the described loaded reactive latex, possibly containing other biological, pharmaceutical or diagnostic components, to the interior of a cell. In RME, the binding of a ligand with a receptor on the surface of a cell can initiate an endocytosis response. Thus, the loaded reactive latex or its conjugate, with associated targeting moiety on surface, can bind on the surface of a cell and subsequently be invigorated and internalized within the cell. A representative, but non-limiting, list of moieties that can be employed as targeting agents useful with the present compositions is selected from the group consisting of proteins, peptides (such as folic acid, cyclic RGD peptide, AHNP peptide), aptomers, antibodies, antibody fragments, small organic molecules, toxins, diptheria toxin, pseudomonas toxin, cholera toxin, ricin, concanavalin A, Rous sarcoma virus, Semliki forest virus, vesicular stomatitis virus, virus fragments, phage, phage fragments, adenovirus, transferrin, low density lipoprotein, transcobalamin, yolk proteins, epidermal growth factor (i.e. human epidermal growth factor), growth hormone, thyroid stimulating hormone, nerve growth factor, calcitonin, glucagon, prolactin, luteinizing hormone, thyroid hormone, platelet derived growth factor, interferon, catecholamines, peptidomimetrics, glycolipids, glycoproteins and polysacchorides. Homologs or fragments of the presented moieties can also be employed. These targeting moieties can be associated with the loaded reactive latex by chemical bonding and be used to direct the loaded latex-conjugate to a target cell, where it can subsequently be internalized. There is no requirement that the entire moiety be used as a targeting moiety. Smaller fragments of these moieties known to interact with a specific receptor or other structure can also be used as a targeting moiety.

An antibody or an antibody fragment represents a class of most universally used targeting moiety that can be employed to enhance the cellular uptake of loaded reactive latex or loaded latex-conjugate. Antibodies may be prepared by any of a variety of techniques known to those of ordinary skill in the art. Antibodies can be made by cell culture techniques, including the generation of monoclonal antibodies or via transfection of antibody genes into suitable bacterial or mammalian cell hosts, in order to produce recombinant antibodies. In one technique, an immunogen comprising the polypeptide is initially injected into any of a wide variety of mammals (e.g., mice, rats, rabbits, sheep or goats). A superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein (“Derivation of specific antibody-producing tissue culture and tumor lines by cell fusion”, Eur. J. Immunol. 1976, 6511-6519), and improvements thereto. Monoclonal antibodies may be isolated from the supernatants of growing hybridoma colonies. In addition, various techniques may be utilized to enhance the yield, such as injection of the hybridoma cell line into the peritoneal cavity of a suitable vertebrate host, such as a mouse. Monoclonal antibodies may then be collected from the ascites fluid or the blood. Contaminants may be removed from the antibodies by conventional techniques, such as chromatography, gel filtration, precipitation, and extraction. The polypeptides may be used in the purification process, for example, an affinity chromatography step.

A number of “humanized” antibody molecules comprising an antigen-binding site derived from a non-human immunoglobulin have been described (Winter et al. Nature 1991, 349:293-299; Lobuglio et al. Proc. Nat. Acad. Sci. USA, 1989, 86, 4220-4224). These “humanized” molecules are designed to minimize unwanted immunological response toward rodent antihuman antibody molecules that limits the duration and effectiveness of therapeutic applications of those moieties in human recipients.

Vitamins, peptides and other essential minerals and nutrients can be utilized as targeting moiety to enhance the intracellular uptake of loaded reactive latex or loaded latex-conjugate. In particular, a vitamin ligand or peptide can be selected from the group consisting of folate, folate receptor-binding analogs of folate, and other folate receptor-binding ligands, biotin, biotin receptor-binding analogs of biotin, and other biotin receptor-binding ligands, anti-HER2/neu peptide (AHNP) for tumor targeting, HER2 receptor (human epidermal growth factor receptor 2), cyclic RGD peptide, riboflavin, riboflavin receptor-binding analogs of riboflavin and other riboflavin receptor-binding ligands, and thiamin, thiamin receptor-binding analogs of thiamin and other thiamin receptor-binding ligands. Additional nutrients believed to trigger receptor mediated endocytosis, and thus also having application in accordance with the presently disclosed method, are carnitine, inositol, lipoic acid, niacin, pantothenic acid, pyridoxal, and ascorbic acid, and the lipid soluble vitamins A, D, E and K. Furthermore, any of the “immunoliposomes” (liposomes having an antibody linked to their surface) described in the prior art are suitable for use with the described loaded reactive latex or loaded latex-conjugates.

Since not all natural cell membranes are over-expressed with biologically active HER2, biotin or folate receptors, use of the described compositions in-vitro on a particular cell line can involve altering or otherwise modifying that cell line first to ensure the presence of biotin, HER2 or folate receptors. Thus, the number of biotin, HER2 or folate receptors on a cell membrane can be increased by growing a cell line on biotin, HER2 or folate deficient substrates, or by expression of an inserted foreign gene for the protein or apoprotein corresponding to the biotin or folate receptor.

RME is not the exclusive method by which the loaded reactive latex or loaded latex-conjugates can be internalized within a cell. Other methods of intracellular uptake that can be exploited by attaching the appropriate entity (e.g. TAT cell penetrating peptide) to a loaded reactive latex or loaded latex-conjugate include the advantageous use of membrane pores. Phagocytotic and pinocytotic mechanisms also offer advantageous mechanisms by which a loaded reactive latex or loaded latex-conjugate can be internalized inside a cell.

The recognition moiety can further comprise a sequence that is subject to enzymatic or electrochemical cleavage. The recognition moiety can thus comprise a sequence that is susceptible to cleavage by enzymes present at various locations inside a cell, such as proteases or restriction endonucleases (e.g. DNAse or RNAse).

A cell surface recognition sequence or moiety is not a requirement. Thus, although a cell surface receptor-targeting moiety can enhance the targeting efficiency, or increase intracellular uptake, there is no requirement that a cell surface targeting moiety be present on the surface of the loaded reactive latex when it can be utilized for cell labeling.

In one embodiment, the loaded reactive latex can contain more than one molecular imaging agent for dual-model or multimodel imaging applications in vitro, in vivo or for diagnostic applications. When so used, the loaded reactive latex may attach with targeting moiety on their surface. Included within the scope of the invention are compositions comprising reactive latex and other suitable imagable moieties by loading, conjugation, or chelating. The nature of the imagable moiety depends on the imaging modality utilized in the diagnosis. The imagable moiety must be capable of detection either directly or indirectly in an in vivo diagnostic imaging procedure, for example, moieties which emit or may be caused to emit detectable radiation (e.g. by radioactive decay, fluorescence excitation or spin resonance excitation), moieties which affect local electromagnetic fields (e.g. paramagnetic, superparamagnetic, ferrimagnetic or ferromagnetic species), moieties which absorb or scatter radiation energy (e.g. chromophores, particles (including gas or liquid containing vesicles), heavy elements and compounds thereof, etc.), and moieties which generate a detectable substance (e.g. gas microbubble generators).

A very wide range of materials detectable by diagnostic imaging modalities is known from the art. Thus, for example, for ultrasound imaging an echogenic material, or a material capable of generating an echogenic material will normally be selected, for X-ray imaging the imagable moieties will generally be or contain a heavy atom (e.g. of atomic weight 38 or above), for magnetic resonance imaging (MRI) the imagable moieties will either be a non zero nuclear spin isotope (such as ¹⁹F) or a material (e.g. iron oxide nanoparticles) having unpaired electron spins and hence paramagnetic, superparamagnetic, ferrimagnetic or ferromagnetic properties, for light imaging the imagable moieties will be a light scatterer (e.g. a colored or uncolored particle), a light absorber or a light emitter, for magnetometric imaging the imagable moieties will have detectable magnetic properties, for electrical impedance imaging the imagable moieties will affect electrical impedance and for scintigraphy, SPECT, PET etc. the imagable moieties will be a radionuclide.

Examples of the suitable imagable moieties are widely known from the diagnostic imaging literature, e.g. magnetic iron oxide particles, gas-containing vesicles, chelated paramagnetic metals (such as Gd, Dy, Mn, Fe etc.). Particularly preferred imagable moieties are: chelated paramagnetic metal ions such as Gd, Dy, Fe, and Mn, especially when chelated by macrocyclic chelant groups (e.g. tetraazacyclododecane chelants such as 1,4,7,10-tetraazacyclododecane-N, N′,N″,N′″-tetraacetic acid (DOTA), 1,4,7,10-tetraazacyclododecane-N,N′,N″-triacetic acid (DO3A), HP-DO3A (10-(2-hydroxypropyl)-1,4,7,10-tetraazacyclododecane-1,4,7triacetic acid) and analogues thereof; or by linker chelant groups such as DTPA (N,N,N′,N″,N″-diethylene-triaminepentaacetic acid (DTPA), DTPA-BMA (N,N,N′,N″,N″-diethylenetriaminepentaacetic acid bismethylamide), DPDP (N,N′-dipyridoxylethylenediamine-N,N′-diacetate-5,5′-bis(phosphate), ethylenediamine-N,N,N′,N′-tetraacetic acid (EDTA), 1-oxa-4,7,10-triazacyclododecane-N,N′, N″-triacetic acid (OTTA), trans(1,2)-cyclohexanodiethylene-triamine-pentaacetic acid (CDTPA), etc; metal radionuclide such as ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce; superparamagnetic iron oxide crystals; chromophores and fluorophores having absorption and/or emission maxima in the range 300-1400 nm, especially 600 nm to 1200 nm, in particular 650 to 1000 nm; vesicles containing fluorinated gases (i.e. containing materials in the gas phase at 37° C. which are fluorine containing, e.g. SF₆ or perfluorinated C₁₋₆ hydrocarbons or other gases and gas precursors listed in WO 97/29783); chelated heavy metal cluster ions (e.g. W or Mo polyoxoanions or the sulphur or mixed oxygen/sulphur analogs); covalently bonded non-metal atoms which are either high atomic number (e.g. iodine) or are radioactive, e.g. ¹²³I , ¹³¹I, etc. atoms; iodinated compound containing vesicles; etc.

Stated generally, the imagable moieties may be (1) a chelatable metal or polyatomic metal-containing ion (i.e. TcO, etc), where the metal is a high atomic number metal (e.g. atomic number greater than 37), a paramagnetic species (e.g. a transition metal or lanthanide), or a radioactive isotope, (2) a covalently bound non-metal species which is an unpaired electron site (e.g. an oxygen or carbon in a persistent free radical), a high atomic number non-metal, or a radioisotope, (3) a polyatomic cluster or crystal containing high atomic number atoms, displaying cooperative magnetic behavior (e.g. superparamagnetism, ferrimagnetism or ferromagnetism) or containing radionuclides, (4) a gas or a gas precursor (i.e. a material or mixture of materials which is gaseous at 37° C.), (5) a chromophore (by which term species which are fluorescent or phosphorescent are included), e.g. an inorganic or organic structure, particularly a complexed metal ion or an organic group having an extensive delocalized electron system, or (6) a structure or group having electrical impedance varying characteristics, e.g. by virtue of an extensive delocalized electron system. Examples of particular imagable moieties are described in more detail below.

Chelated metal imagable moieties: Metal Radionuclides, Paramagnetic metal ions, Fluorescent metal ions, Heavy metal ions and cluster ions. Preferred metal radionuclides include ⁹⁰Y, ^{99m}Tc, ¹¹¹In, ⁴⁷Sc, ⁶⁷Ga, ⁵¹Cr, ^{177m}Sn, ⁶⁷Cu, ¹⁶⁷Tm, ⁹⁷Ru, ¹⁸⁸Re, ¹⁷⁷Lu, ¹⁹⁹Au, ²⁰³Pb and ¹⁴¹Ce; Preferred paramagnetic metal ions include ions of transition and lanthanide metals (e.g. metals having atomic numbers of 6 to 9, 21-29, 42, 43, 44, or 57-71), in particular ions of Cr, V, Mn, Fe, Co, Ni, Cu, La, Ce, Pr, Nd, Pm, Sm, Eu, Gd, Th, Dy, Ho, Er, Tm, Yb and Lu, especially of Mn, Cr, Fe, Gd and Dy, more especially Gd. Preferred fluorescent metal ions include lanthanides, in particular La, Ce, Pr, Nd, Pm, Sm, Eu, Gd, Th, Dy, Ho, Er, Tm, Yb, and Lu—Eu is especially preferred. Preferred heavy metal-containing imagable moieties may include atoms of Mo, Bi, Si, and W, and in particular may be polyatomic cluster ions (e.g. Bi compounds and W and Mo oxides). The metal ions are desirably chelated by chelant groups in particular linear, macrocyclic, terpyridine and N2S2 chelants, such as for example ethylenediamine-N,N,N′,N′-tetraacetic acid (EDTA); N,N, N′,N″,N″-diethylene-triaminepentaacetic acid (DTPA); 1,4,7,10-tetraazacyclododecane-N,N′,N″,N′″-tetraacetic acid (DOTA); 1,4,7,10-tetraazacyclododecaneN,N′,N″-triacetic acid (DO3A); 1-oxa-4,7,10-triazacyclododecane-N,N′,N″-triacetic acid (OTTA); trans(1,2)-cyclohexanodiethylene-triamine-pentaacetic acid (CDTPA), TMT (terpyridine-bis(methylenaminetetraacetic acid)

Further examples of suitable chelant groups are disclosed in U.S. Pat. No. 4,647,447; U.S. Pat. No.5,367,080; and U.S. Pat. No.5,364,613. The imagable moiety may contain one or more such chelant groups, if desired metallated by more than one metal species (e.g. so as to provide the imagable moieties detectable in different imaging modalities). Particularly where the metal is non-radioactive, it is preferred that a polychelant moiety is used.

A chelant or chelating group as referred to herein may comprise the residue of one or more of a wide variety of chelating agents that can complex a metal ion or a polyatomic ion (e.g. TcO).

As is well known, a chelating agent is a compound containing donor atoms that can combine by coordinate bonding with a metal atom to form a cyclic structure called a chelation complex or chelate. The reside of a suitable chelating agent can be selected from polyphosphates, such as sodium tripolyphosphate and hexametaphosphoric acid; aminocarboxylic acids, such as EDTA (ethylenediaminetetraacetic acid), N-(2-hydroxy)ethylenediaminetriacetic acid, nitrilotriacetic acid, N,N-di(2-hydroxyethyl)glycine, ethylenebis(hydroxyphenylglycine) and diethylenetriamine pentacetic acid; 1,3-diketones, such as acetylacetone, trifluoroacetylacetone, and thenoyltrifluoroacetone; hydroxycarboxylic acids, such as tartaric acid, citric acid, gluconic acid, and 5-sulfosalicyclic acid; polyamines, such as ethylenediamine, diethylenetriamine, triethylenetetraamine, and triaminotriethylamine; aminoalcohols, such as triethanolamine and N-(2-hydroxyethyl)ethylenediamine; aromatic heterocyclic bases, such as 2,21-diimidazole, picoline amine, dipicoline amine and 1,10-phenanthroline; phenols, such as salicylaldehyde, disulfopyrocatechol, and chromotropic acid; aminophenols, such as 8-hydroxyquinoline and oximesulfonic acid; oximes, such as dimethylglyoxime and salicylaldoxime; peptides containing proximal chelating functionality such as polycysteine, polyhistidine, polyaspartic acid, polyglutamic acid, or combinations of such amino acids; Schiff bases, such as disalicylaldehyde 1,2-propylenediimine; tetrapyrroles, such as tetraphenylporphin and phthalocyanine; sulfur compounds, such as toluenedithiol, meso-2,3-dimercaptosuccinic acid, dimercaptopropanol, thioglycolic acid, potassium ethyl xanthate, sodium diethyldithiocarbamate, dithizone, diethyl dithiophosphoric acid, and thiourea; synthetic macrocyclic compounds, such as dibenzo [18-crown-6, (CH₃)₆-[14]-4,11]-diene-N₄, and (2.2.2-cryptate); phosphonic acids, such as nitrilotrimethylene-phosphonic acid, ethylenediaminetetra(methylenephosphonic acid), and hydroxyethylidenediphosphonic acid, or combinations of two or more of the above agents. The residue of a suitable chelating agent preferably comprises a polycarboxylic acid group and preferred examples include: ethylenediamine-N,N,N′,N′-tetraacetic acid (EDTA); N,N,N′,N″,N″-diethylene-triaminepentaacetic acid (DTPA); 1,4,7,10-tetraazacyclododecane-N, N′,N″,N′″-tetraacetic acid (DOTA); 1,4,7,10-tetraazacyclododecaneN,N′,N″-triacetic acid (D03A); 1-oxa-4,7,10-triazacyclododecane-N,N′,N″-triacetic acid (OTTA); trans(1,2)-cyclohexanodiethylene-triamine-pentaacetic acid (CDTPA),other suitable residues of chelating agents comprise proteins modified for the chelation of metals such as technetium and rhenium as described in U.S. Pat. No. 5,078,985, the disclosure of which is hereby incorporated by reference.

Metals can be incorporated into a chelant moiety by any one of three general methods: direct incorporation, template synthesis and/or transmetallation. In one embodiment direct incorporation is utilized.

Thus, it is desirable that the metal ion be easily complexed to the chelating agent, for example, by merely exposing or mixing an aqueous solution of the chelating agent-containing moiety with a metal salt in an aqueous solution preferably having a pH in the range of about 4 to about 11. The salt can be any salt, but preferably the salt is a water soluble salt of the metal such as a halogen salt, and more preferably such salts are selected so as not to interfere with the binding of the metal ion with the chelating agent. The chelating agent-containing moiety is preferably in aqueous solution at a pH of between about 5 and about 9, more preferably between pH about 6 to about 8. The chelating agent-containing borate to produce the optimum pH. Preferably, the buffer salts are selected so as not to interfere with the subsequent binding of the metal ion to the chelating agent.

Where the imagable moiety contains a single chelant, that chelant may be attached directly to the nanolatex, e.g. via one of the metal coordinating groups of the chelant which may form an ester, amide, thioester or thioamide bond with an amine, thiol or hydroxyl group on the reactive nanolatex. Alternatively the reactive nanolatex and chelant may be directly linked via a functionality attached to the chelant backbone, e.g. a CH₂-phenyl-NCS group attached to a ring carbon of DOTA and DTPA as proposed by Meares et al. in JACS 110:6266-6267(1988), or indirectly via a homo or hetero-bifunctional linker, e.g. a bis amine, bis epoxide, diol, diacid, difunctionalized PEG, etc.

Non-metal atomic imagable moiety: Preferred non-metal atomic imagable moieties include radioisotopes such as ¹²³I and ¹³¹I as well as non zero nuclear spin atoms such as ¹⁸F, and heavy atoms such as I. Such imagable moieties, preferably a plurality thereof, e.g. 2 to 200, may be covalently bonded to a linker backbone, either directly using conventional chemical synthesis techniques or via a supporting group.

Biomedical Application: The water dispersible loaded reactive latex for mono-model, dual-model or multi-model imaging may also be useful in other biomedical applications, including, but not limited to, tomographic imaging of organs, monitoring of organ functions, coronary angiography, fluorescence endoscopy, imaging and determining efficacy of drug delivery and therapy, detection and diagnostics of diseases (e.g. cancers, heart disease, diabetes, stroke, or other), laser assisted guided surgery, photoacoustic methods, and sonofluorescent methods.

The compositions can be formulated into diagnostic compositions for enteral, parenteral, oral, trandermal or transmucosal administration. These compositions contain an effective amount of the imaging agents (e.g. dyes) along with conventional pharmaceutical carriers and excipients appropriate for the type of administration contemplated. Parenteral compositions may be injected directly or mixed with a large volume parenteral composition for systemic administration. Formulations for enteral administration may vary widely, as is well known in the art. In general, such enteral formulations are liquids which include an effective amount of the imaging agents in aqueous solution or suspension. Such enteral compositions may optionally include buffers, surfactants, thixotropic agents, and the like. Compositions for oral administration may also contain flavoring agents and other ingredients for enhancing their organoleptic qualities.

The diagnostic compositions are administered in doses effective to achieve the desired enhancement. Such doses may vary widely, depending upon the particular imaging probes (e.g. dyes) employed, the organs or tissues which are the subject of the imaging procedure, the imaging equipment being used, and the like. The compositions may be administered to a patient, typically a warm-blooded animal, either systemically or locally to the organ or tissue to be imaged, and the patient then subjected to the imaging procedure.

The preferred administration techniques include parenteral administration, intravenous administration and infusion directly into any desired target tissue, including but not limited to a solid tumor or other neoplastic tissue. Purification can be achieved by employing a final purification step, which disposes the loaded reactive latex or loaded latex-conjugate composition in a medium comprising a suitable pharmaceutical composition. Suitable pharmaceutical compositions generally comprise an amount of the desired loaded reactive latex or loaded latex-conjugate with active agent in accordance with the dosage information (which is determined on a case-by-case basis). The described nanolatex particles are admixed with an acceptable pharmaceutical diluent or excipient, such as a sterile aqueous solution, to give an appropriate final concentration. Such formulations can typically include buffers such as phosphate buffered saline (PBS), or additional additives such as pharmaceutical excipients, stabilizing agents such as BSA or HSA, or salts such as sodium chloride.

For parenteral administration it is generally desirable to further render such compositions pharmaceutically acceptable by insuring their sterility, non-immunogenicity and non-pyrogenicity. Such techniques are generally well known in the art. Moreover, for human administration, preparations should meet sterility, pyrogenicity, and general safety and purity standards as required by FDA Office of Biological Standards. When the described loaded latex or loaded latex-conjugate composition is being introduced into cells suspended in a cell culture, it is sufficient to incubate the cells together with the nanoparticle in an appropriate growth media, for example Luria broth (LB) or a suitable cell culture medium. Although other introduction methods are possible, these introduction treatments are preferable and can be performed without regard for the entities present on the surface of a loaded latex carrier.

The loaded nanolatex-conjugates, whether for single or multicolor detection systems, are combined with a sample thought to contain target materials. Typically the sample is incubated with an aqueous dispersion of the loaded nanolatex-conjugates. Where a single color detection system is used, the aqueous dispersion contains substantially identical loaded nanolatex-conjugates. Where a multicolor detection system is used, the aqueous dispersion contains a number of detectable different loaded nanolatex-conjugates. In each case, the loaded nanolatex-conjugates are specific for a particular target or combination of specific targets.

Prior to combination with the loaded nanolatex-conjugates, the sample is prepared in a way that makes the target materials in the sample accessible to the latex-based imaging probes. The target materials may require purification or separation prior to labeling or detection. For example, the sample may contain purified nucleic acids, proteins, receptors, peptides, bioactive ligands, or carbohydrates, either in mixtures or individual nucleic acid, protein, peptide, or carbohydrate species; the sample may contain nucleic acids, proteins, or carbohydrates in lysed cells along with other cellular components; or the sample may contain nucleic acids, proteins, or carbohydrates in substantially whole, permeabilized cells. Preparation of the sample will depend on the way the target materials are contained in the sample. When the sample contains cellular nucleic acids (such as chromosomal or plasmid borne genes within cells, RNA or DNA, viruses or mycoplasma infecting cells, or intracellular RNA) or proteins, reparation of the sample involves lysing or permeabilizing the cell, in addition to the denaturation and neutralization already described.

Following the labeling of the sample with the loaded nanolatex-conjugates, unbound loaded nanolatex-conjugates are optionally removed from the sample by conventional methods such as washing.

For detection of the target materials, the sample is illuminated with means for emissive fluorescence from the loaded nanolatex-conjugates. Typically a source of excitation energy emitting within the excitation range of the loaded latex-conjugates is used. Fluorescence resulting from the loaded latex-conjugates that have formed a complex with the target materials can be used to detect the presence, location, or quantity of target materials.

Fluorescence from the loaded nanolatex-conjugates can be visualized by a variety of imaging techniques, including ordinary optical or fluorescence microscopy and confocal laser scanning fluorescence microscopy and CCD cameras. Three-dimensional imaging resolution techniques in confocal microscopy utilize knowledge of the microscope's point spread function (image of a point source) to place out-of-focus light in its proper perspective. Multiple labeled target materials are optionally resolved spatially, chronologically, by size, or using detectably different spectral characteristics (including excitation and emission maxima, fluorescence intensity, or combinations thereof). Typically, multiple labeled target materials are resolved using different loaded nanolatex-conjugates with distinct spectral characteristics for each target material. Alternatively, the loaded latex-conjugates are the same but the samples are labeled and viewed sequentially or spatially separated. If there is no need or desire to resolve multiple targets, as in wide scale screening (e.g. pan-viral or bacterial contamination screening), loaded latex-conjugates containing multiple target complements need not be separately applied to samples. Therapeutic agents are materials which affect enhance or inhibit cellular function, blood flow, or biodistribution, or bioabsorbtion. Examples include pharmaceutical drugs for cancer, heart disease, genetic disorders, bacterial and viral infection and many other disorders.

Other useful materials to conjugate include: PEG-peptide, PEG-protein, PEG-enzyme inhibitor, PEG-oligosaccharide, PEG-polygosaccharide, PEG-hormone, PEG-dextran, PEG-oligonucleotide, PEG-carbohydrate, PEG-neurotransmitter, PEG-hapten, and PEG-carotinoid. In one embodiment the PEG is functionalized with mixtures of these materials to improve effectiveness.

The following is a list of preferred linking polymers, but is not intended to an exhaustive and complete list of all linking polymers according to the present invention: In one method of use, multiple linking polymers are attached to a nanolatex. For example, a first mixture of monomer(s) of interest, the linking polymer, and initiator is prepared in water. The first mixture was added to the second mixture of additional initiator and reacted, after which, additional initiator may be added to produce a nanolatex composition. In another preferred method of use, multiple linking polymers are attached to a nanolatex. A mixture of monomers, linking polymer containing macromonomers, initiator, surfactant, and buffer was prepared in water. The mixture is added to an aqueous solution of initiator, surfactant and buffer and reacted to produce a nanolatex particle according to the present invention.

In general, the derivatization is performed under any suitable condition used to react a biologically active substance with an activated water soluble linking polymer molecule. In general, the optimal reaction conditions for the acylation reactions will be determined case-by-case based on known parameters and the desired result. For example, the larger the ratio of PEG: protein, the greater the percentage of polypegylated product. One may choose to prepare a mixture of linking polymer/polypeptide conjugate molecules by acylation and/or alkylation methods, and the advantage provided herein is that one may select the proportion of monopolymer/polypeptide conjugate to include in the mixture.

The latexes useful in this invention may be prepared by any method known in the art for preparing particles of 5-100 nm in mean diameter. Especially useful methods include emulsion and miniemulsion polymerization. Such techniques are reviewed in “Suspension, Emulsion, and Dispersion Polymerization: a Methodological Survey” Colloid. Polym. Sci. vol. 270, p. 717-732, 1992 and in Lovell, P. A.; El-Aaser, M. S. “Emulsion Polymerization and Emulsion Polymers”,Wiley: Chichester, 1997. An alternate method involves intramolecularly crosslinking individual polymer chains to form very small particles. This method is further described in U.S. Pat. No. 6,890,703.

Dyes useful for this invention are fluorescent dyes, not limited to hydrophobic dyes, with emissive fluorescence from 400 to 1000 nm. Classes of dyes include, but are not necessarily limited to oxonol, pyrylium, Squaric, croconic, rodizonic, polyazaindacenes or coumarins, scintillation dyes (usually oxazoles and oxadiazoles), aryl- and heteroaryl-substituted polyolefins (C₂-C₈ olefin portion), merocyanines, carbocyanines, phthalocyanines, oxazines, carbostyryl, porphyrin dyes, dipyrrometheneboron difluoride dyes aza-dipyrrometheneboron difluoride dyes and oxazine dyes. Commercially available fluorescent dyes are listed in Table 1 and generic structures are shown in Table 2. Preferred dyes are carbocyanine, phthalocyanine, or aza-dipyrrometheneboron difluoride.

TABLE 1
Commercially available fluorescent dyes.

5-Amino-9-diethyliminobenzo(a)phenoxazonium Perchlorate
7-Amino-4-methylcarbostyryl
7-Amino-4-methylcoumarin
7-Amino-4-trifluoromethylcoumarin
3-(2′-Benzimidazolyl)-7-N,N-diethylaminocoumarin
3-(2′-Benzothiazolyl)-7-diethylaminocoumarin
2-(4-Biphenylyl)-5-(4-t-butylphenyl)-1,3,4-oxadiazole
2-(4-Biphenylyl)-5-phenyl-1,3,4-oxadiazole
2-(4-Biphenyl)-6-phenylbenzoxazole-1,3
2,5-Bis-(4-biphenylyl)-1,3,4-oxadiazole
2,5-Bis-(4-biphenylyl)-oxazole
4,4′″-Bis-(2-butyloctyloxy)-p-quaterphenyl
p-Bis(o-methylstyryl)-benzene
5,9-Diaminobenzo(a)phenoxazonium Perchlorate
4-Dicyanomethylene-2-methyl-6-(p-dimethylaminostyryl)-4H-pyran
1,1′-Diethyl-2,2′-carbocyanine Iodide
1,1′-Diethyl-4,4′-carbocyanine Iodide
3,3′-Diethyl-4,4′,5,5′-dibenzothiatricarbocyanine Iodide
1,1′-Diethyl-4,4′-dicarbocyanine Iodide
1,1′-Diethyl-2,2′-dicarbocyanine Iodide
3,3′-Diethyl-9,11-neopentylenethiatricarbocyanine Iodide
1,3′-Diethyl-4,2′-quinolyloxacarbocyanine Iodide
1,3′-Diethyl-4,2′-quinolylthiacarbocyanine Iodide
3-Diethylamino-7-diethyliminophenoxazonium Perchlorate
7-Diethylamino-4-methylcoumarin
7-Diethylamino-4-trifluoromethylcoumarin
7-Diethylaminocoumarin
3,3′-Diethyloxadicarbocyanine Iodide
3,3′-Diethylthiacarbocyanine Iodide
3,3′-Diethylthiadicarbocyanine Iodide
3,3′-Diethylthiatricarbocyanine Iodide
4,6-Dimethyl-7-ethylaminocoumarin
2,2′″-Dimethyl-p-quaterphenyl
2,2″-Dimethyl-p-terphenyl
7-Dimethylamino-1-methyl-4-methoxy-8-azaquinolone-2
7-Dimethylamino-4-methylquinolone-2
7-Dimethylamino-4-trifluoromethylcoumarin
2-(4-(4-Dimethylaminophenyl)-1,3-butadienyl)-3-ethylbenzothiazolium
Perchlorate
2-(6-(p-Dimethylaminophenyl)-2,4-neopentylene-1,3,5-hexatrienyl)-3-
methylbenzothiazolium Perchlorate
2-(4-(p-Dimethylaminophenyl)-1,3-butadienyl)-1,3,3-trimethyl-3H-
indolium
Perchlorate
3,3′-Dimethyloxatricarbocyanine Iodide
2,5-Diphenylfuran
2,5-Diphenyloxazole
4,4′-Diphenylstilbene
1-Ethyl-4-(4-(p-Dimethylaminophenyl)-1,3-butadienyl)-pyridinium
Perchlorate
1-Ethyl-2-(4-(p-Dimethylaminophenyl)-1,3-butadienyl)-pyridinium
Perchlorate
1-Ethyl-4-(4-(p-Dimethylaminophenyl)-1,3-butadienyl)-quinolium
Perchlorate
3-Ethylamino-7-ethylimino-2,8-dimethylphenoxazin-5-ium Perchlorate
9-Ethylamino-5-ethylamino-10-methyl-5H-benzo(a)phenoxazonium
Perchlorate
7-Ethylamino-6-methyl-4-trifluoromethylcoumarin
7-Ethylamino-4-trifluoromethylcoumarin
1,1′,3,3,3′,3′-Hexamethyl-4,4′,5,5′-dibenzo-2,2′-
indotricarboccyanine Iodide
1,1′,3,3,3′,3′-Hexamethylindodicarbocyanine Iodide
1,1′,3,3,3′,3′-Hexamethylindotricarbocyanine Iodide
2-Methyl-5-t-butyl-p-quaterphenyl
3-(2′-N-Methylbenzimidazolyl)-7-N,N-diethylaminocoumarin
2-(1-Naphthyl)-5-phenyloxazole
2,2′-p-Phenylen-bis(5-phenyloxazole)
3,5,3′″″,5′″″-Tetra-t-butyl-p-sexiphenyl
3,5,3″″,5″″-Tetra-t-butyl-p-quinquephenyl
2,3,5,6-1H,4H-Tetrahydro-9-acetylquinolizino-<9,9a,1-gh> coumarin
2,3,5,6-1H,4H-Tetrahydro-9-carboethoxyquinolizino-<9,9a,1-
gh> coumarin
2,3,5,6-1H,4H-Tetrahydro-8-methylquinolizino-<9,9a,1-> coumarin
2,3,5,6-1H,4H-Tetrahydro-9-(3-pyridyl)-quinolizino-<9,9a,1-
gh> coumarin
2,3,5,6-1H,4H-Tetrahydro-8-trifluoromethylquinolizino-<9,9a,1-
gh> coumarin
2,3,5,6-1H,4H-Tetrahydroquinolizino-<9,9a,1-gh> coumarin
3,3′,2″,3′″-Tetramethyl-p-quaterphenyl
2,5,2″″,5″″-Tetramethyl-p-quinquephenyl
P-terphenyl
P-quaterphenyl
Nile Red
Rhodamine 700
Oxazine 750
Rhodamine 800
IR 125
IR 144
IR 140
IR 132
IR 26
IR 5
Diphenylhexatriene
Diphenylbutadiene
Tetraphenylbutadiene
Naphthalene
Anthracene
Pyrene
Chrysene
Rubrene
Coronene
Phenanthrene
Fluorene
Aluminum phthalocyanine
Platinum octaethylporphyrin

TABLE 2
Illustrative Examples of Fluorescent Dyes

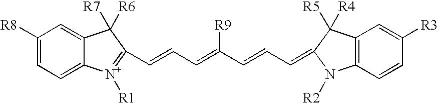

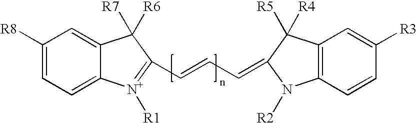

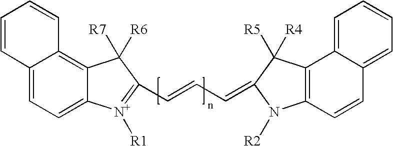

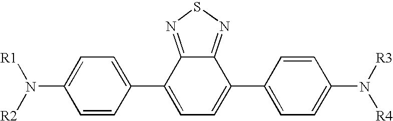

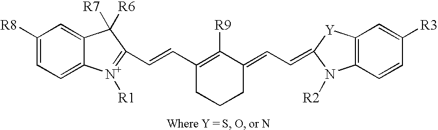

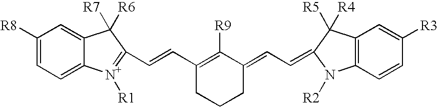

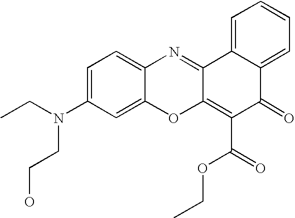

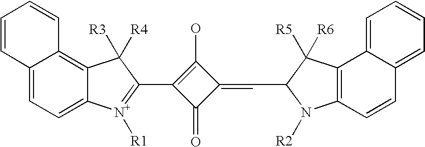

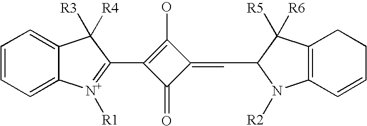

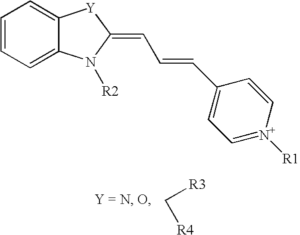

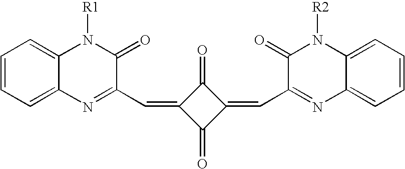

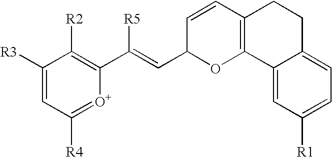

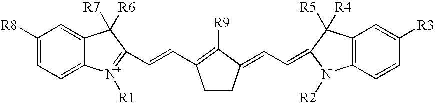

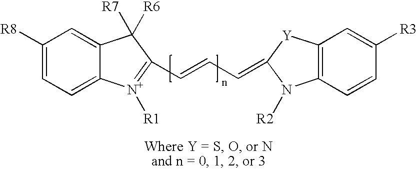

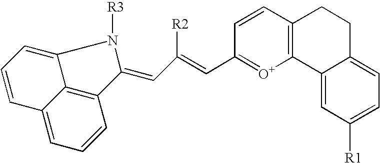

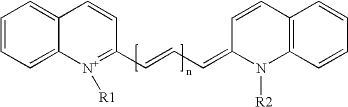

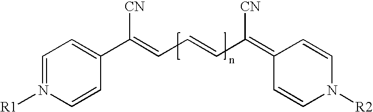

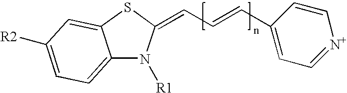

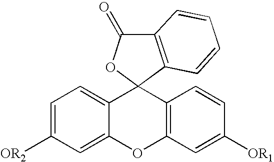

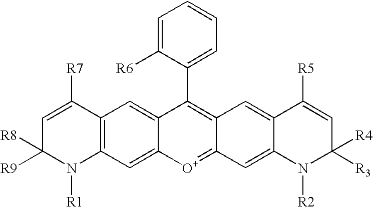

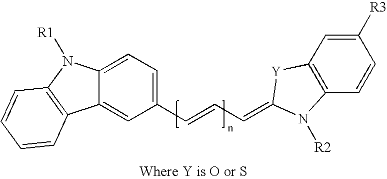

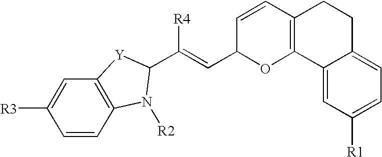

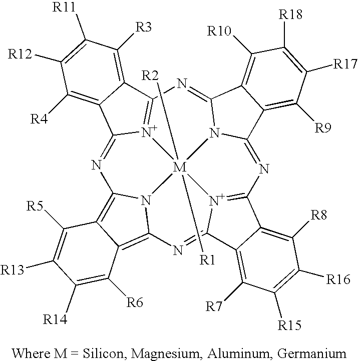

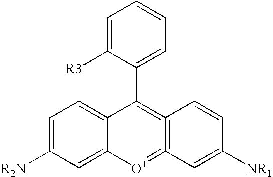

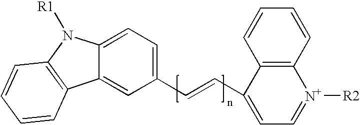

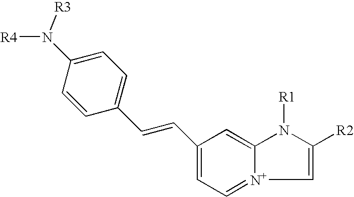

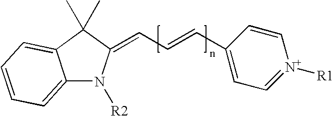

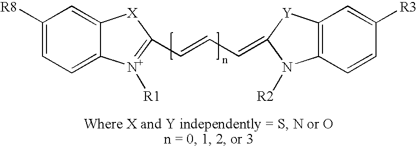

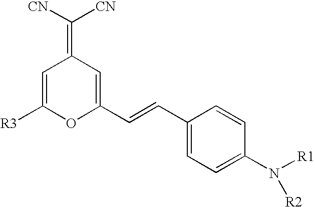

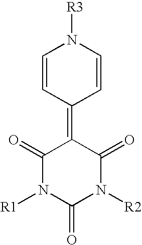

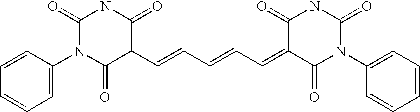

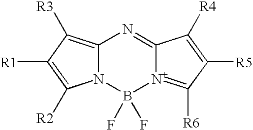

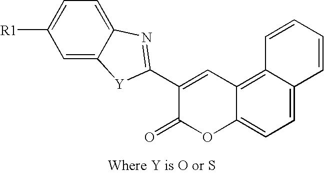

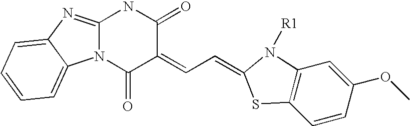

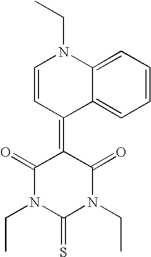

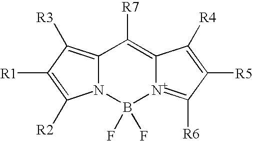

R1-R18 are independently hydrogen, alkyl, alkoxy, alkenyl, cycloalkyl, arylalkyl, acyl, heteroaryl, or halogen, amino or substituted amino.

The fluorescent dyes are loaded in the reactive nanolatex and are preferably solvent soluble. When the dyes are loaded into the reactive latex particles that are well dispersed in aqueous solution, a boost is observed in quantum yield of fluorescence from the loaded reactive nanolatex as compared to the quantum yield of the dye in aqueous solvent.

The fluorescent dye and other imaging agents are loaded into the latex by a variety of known methods. For example, a solution of the dye or other imaging agent in a water-miscible organic solvent (e.g. tetrahydrofuran (THF), acetone, methanol, dimethyl sulfoxide (DMSO), dimethylformamide (DMF), or their mixture) can be mixed with the latex, and then the solvent can be removed by evaporation, dilution with water, or dialysis, as further described in U.S. Pat. No. 6,706,460, U.S. Pat. No. 4,368,258, U.S. Pat. No. 4,199,363 and U.S. Pat. No. 6,964,844. A solution of the dye in a water-immiscible organic solvent can be combined with the aqueous latex and the mixture subjected to high shear mixing, as described in U.S. Pat. No.5,594,047. Alternately, the dye can be incorporated during the preparation of the latex. Such a method is described in Journal of Polymer Science Part A: Polymer Chemistry, Vol. 33, p. 2961-2968, 1995 and in Colloid and Polymer Science, vol. 282, p. 119-126, 2003.

The loaded reactive latex particle may be used as an imaging probe for use in animals, as well as other physiological systems. The particle may be used as a diagnostic contrast element or in other in vitro/in vivo, physiological imaging applications. Preferably, the particle is provided in an aqueous, biocompatible dispersion.

The described composition can further comprise a biological, pharmaceutical or diagnostic component that includes a targeting moiety that recognizes the specific target cell or other target biological molecules. As used herein “target cells” refers to healthy cells, disease cells (e.g. various cancer cells), stem cell, mammalian cell, or plant cells. “Target biological molecules” include, but not limited to, antibodies, antibody fragments or subdomains, peptides, polypeptides, bioactive ligands, proteins, protein fragments, nucleic acids, or any essential metabolites.

A representative, but non-limiting, list of moieties suitable as targeting agents useful with the present compositions is selected from the group consisting of proteins, peptides (e.g. tumor targeting peptides), ligands, aptomers, polypeptides, small organic molecules, toxins, diptheria toxin, pseudomonas toxin, cholera toxin, ricin, concanavalin A, Rous sarcoma virus, Semliki forest virus, vesicular stomatitis virus, adenovirus, phages, phage fragments, transferrin, low density lipoprotein, transcobalamin, yolk proteins, epidermal growth factor, growth hormone, thyroid stimulating hormone, nerve growth factor, calcitonin, glucagon, prolactin, luteinizing hormone, thyroid hormone, platelet derived growth factor, interferon, catecholamines, peptidomimetrics, glycolipids, glycoproteins and polysacchorides. Homologs or fragments of the presented moieties can also be employed. These targeting moieties can be associated with a nanoparticulate and be used to direct the nanoparticle to bind a chosen target. There is no requirement that the entire moiety be used as a targeting moiety. Smaller fragments or sequences of these moieties known to interact with a specific receptor or other structure can also be used as a targeting moiety.

An antibody or an antibody fragment represents a class of most universally used targeting moiety that can be linked to a reactive nanolatex. Antibodies may be prepared by any of a variety of techniques known to those of ordinary skill in the art. See, e.g., Harlow and Lane, Antibodies: A Laboratory Manual, Cold Spring Harbor Laboratory, 1988. Antibodies can be produced by cell culture techniques, including the generation of monoclonal antibodies or via transfection of antibody genes into suitable bacterial or mammalian cell hosts, in order to allow for the production of recombinant antibodies. In one technique, an immunogen comprising the polypeptide is initially injected into any of a wide variety of mammals (e.g., mice, rats, rabbits, sheep or goats). A superior immune response may be elicited if the polypeptide is joined to a carrier protein, such as bovine serum albumin or keyhole limpet hemocyanin. The immunogen is injected into the animal host, preferably according to a predetermined schedule incorporating one or more booster immunizations, and the animals are bled periodically. Polyclonal antibodies specific for the polypeptide may then be purified from such antisera by, for example, affinity chromatography using the polypeptide coupled to a suitable solid support.

Monoclonal antibodies specific for an antigenic polypeptide of interest may be prepared, for example, using the technique of Kohler and Milstein, Eur. J. Immunol, 6:511-519, 1976, and improvements thereto.

Monoclonal antibodies may be isolated from the supernatants of growing hybridoma colonies. In addition, various techniques may be employed to enhance the yield, such as injection of the hybridoma cell line into the peritoneal cavity of a suitable vertebrate host, such as a mouse. Monoclonal antibodies may then be harvested from the fluid or the blood. Contaminants may be removed from the antibodies by conventional techniques, such as chromatography, gel filtration, precipitation, and extraction. The polypeptides of this invention may be used in the purification process in, for example, an affinity chromatography step.

A number of “humanized” antibody molecules comprising an antigen-binding site derived from a non-human immunoglobulin have been described (Winter et al. (1991) Nature 349:293-299; Lobuglio et al. (1989) Proc. Nat. Acad. Sci. USA 86:4220-4224). These “humanized” molecules are designed to minimize unwanted immunological response toward rodent antihuman antibody molecules that limits the duration and effectiveness of therapeutic applications of those moieties in human recipients.

The recognition moiety can further comprise a sequence of peptides or nucleic acids that can be recognized by a select target. The peptides and nucleic acids can be selected from a sequence known in the art for their ability to bind to a chosen target, or to be selected from combinatorial peptide or nucleic acid libraries for their ability to bind a chosen target.

Vitamins, peptides and other essential minerals and nutrients can be utilized as targeting moiety to enhance the binding of nanolatex particle to a target. In particular, a vitamin ligand can be selected from the group consisting of folate, folate receptor-binding analogs of folate and other folate receptor-binding ligands, cyclic RGD tumor targeting peptide, anti-HER2 neu peptide and other HER2 receptor binding ligands, biotin, biotin receptor-binding analogs of biotin and other biotin receptor-binding ligands, riboflavin, riboflavin receptor-binding analogs of riboflavin and other riboflavin receptor-binding ligands, avidin, netravidin, streptavidin and their receptors, and thiamin, thiamin receptor-binding analogs of thiamin and other thiamin receptor-binding ligands.

The recognition moiety can further comprise a sequence that is subject to enzymatic or electrochemical cleavage. The recognition moiety can thus comprise a sequence that is susceptible to cleavage by enzymes present at various locations inside a cell, such as proteases or restriction endonucleases (e.g. DNAse or RNAse).

For cell targeting, a cell surface recognition sequence is not a must-have requirement. Thus, although a cell surface receptor targeting moiety can be useful for targeting a given cell type, or for inducing the association of a described nanoparticle with a cell surface, there is no requirement that a cell surface receptor targeting moiety be present on the surface of a reactive nanolatex particle.

To assemble the biological, pharmaceutical or diagnostic components to a described nanoparticulate carrier, the components can be associated with the nanoparticular carrier through a linkage. By “associated with”, it is meant that the component is carried by the nanoparticle, for example the surface of the nanoparticle. The component can be incorporated in the particle by non-covalently link, only by physically encapsulation. A preferred method of associating the component is by covalent bonding through the reactive fluoro-nitro-benzoyl group on the surface.

Generally, any manner of forming a linkage between a targeting moiety of interest and a nanolatex particulate carrier can be utilized. This can include covalent, ionic, or hydrogen bonding of the ligand to the exogenous molecule, either directly or indirectly via a linking group. The linkage is typically formed by covalent bonding of the targeting moiety, biological, pharmaceutical or diagnostic component to the nanoparticle carrier through the formation of imino, amide, or ester between fluoro-nitro-benzoyl, amine (e.g. primary or secondary amine), acid, aldehyde, hydroxy, or hydrazo groups on the respective components of the complex. Art-recognized biologically labile covalent linkages such as imino bonds and so-called “active” esters having the linkage —COOCH, —O—O— or —COOCH are preferred. Hydrogen bonding, e.g., that occurring between complementary strands of nucleic acids, can also be used for linkage formation.

In a one embodiment, the targeting moiety is covalently attached to the reactive group at the end of the polyethylene glycol macromonomer, especially fluoro-nitro-benzoyl reactive group. The covalent linkage used will be dependent on the reactive group at the end of the polyethylene glycol.

The following examples are provided to illustrate of suitable dyes.

TABLE 3
Structures of dyes.

| | |
|---|---|
| Dye 1 | 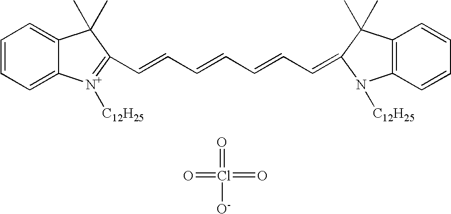 |
| Dye 2 | 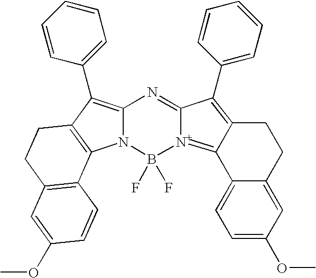 |
| Dye 3 | 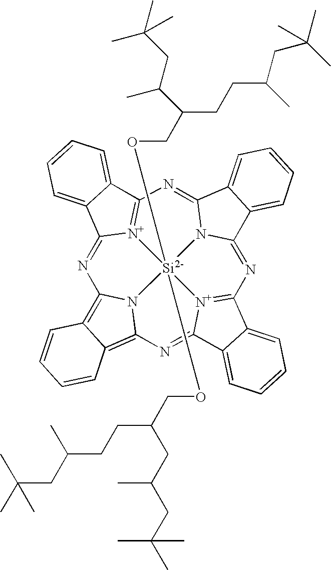 |
| Dye 4 | 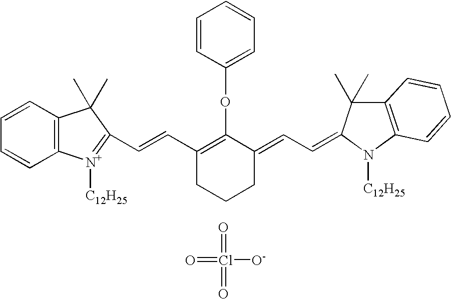 |
| Dye 5 | 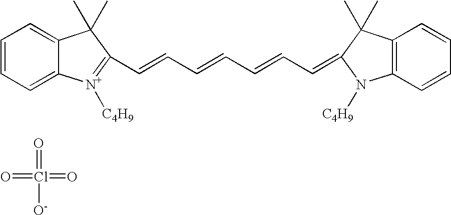 |
| Dye 6 | 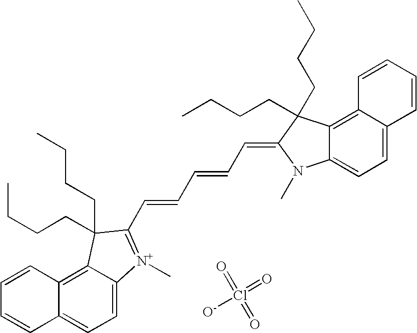 |
| Dye 7 | 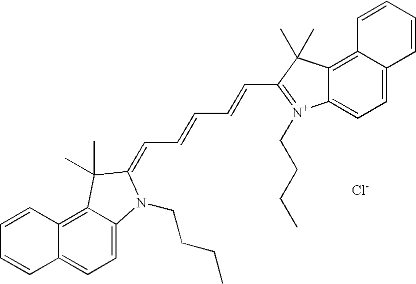 |
| Dye 8 | 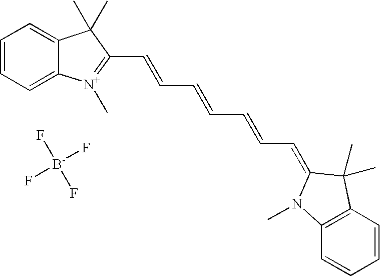 |
| Dye 9* | 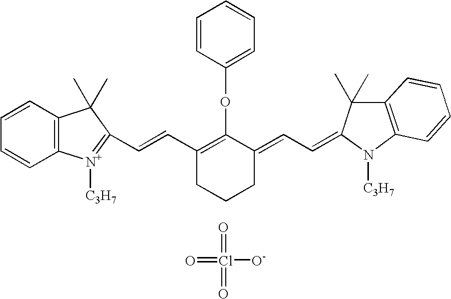 |
| Dye 10 | 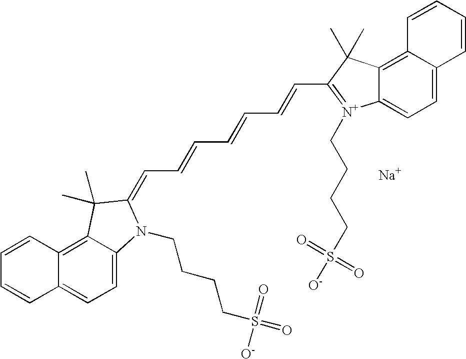 |
| Dye 11 | 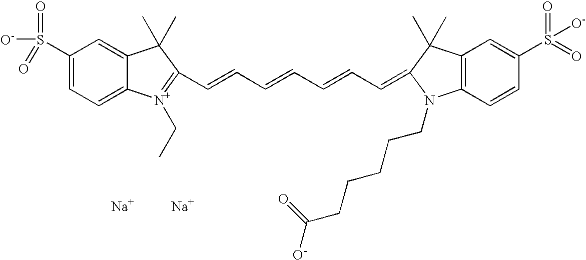 |
| Dye 12** | 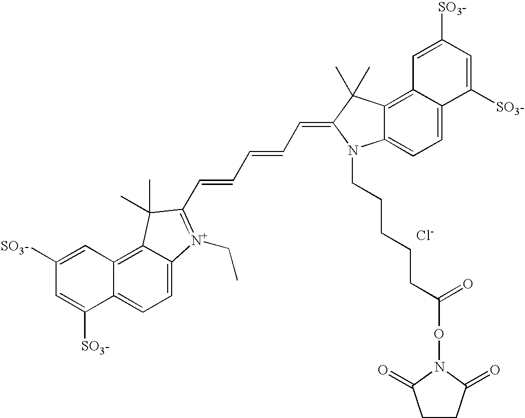 |

*Available from Aldrich Chemicals
**Available from GE Healthcare/Amersham Biosciences

### General Synthetic Procedure 1 for dye 1, dye 5, dye 8, and dye 10.

To a mixture of 3H-Indolium salt (2 eqv.) and N-(5-(phenylamino)-2,4-pentadienylidene)-benzenamine monohydrochloride (the dianil)(1 eqv.) in acetonitrile was added acetic anhydride and triethylamine (1.5 eqv.). The mixture was heated at reflux for 5˜25 min. The resulting mixture was then cooled to room temperature and poured to either water or ether to obtain the crude product, which was further purified either by silica-gel chromatography, or by recrystallization or by reverse phase HPLC.

### EXAMPLE 1

### Dye Synthesis for Preparation of Dye 1

This dye was prepared following the general procedure described above, using 2,3,3-trimethyl-1-octadecyl-3H-Indolium perchlorate (4.28 g, 10 mmol) and the dianil (1.4 g, 5 mmol) in 40 mL of acetic anhydride containing triethylamine (1.5 g, 15 mmoles). The reaction time was 5 minutes. The reaction was cooled to 25 degrees and poured into 2 liters of ice water with vigorous stirring. The water was decanted and the oil was dissolved in 100 mL of 80/20 dichlomethane/methanol mixture. The material was chromatographed on a silica gel column eluting with 80/20 dichlomethane/methanol mixture. Evaporation of the solvent after drying with anhydrous magnesium sulfate afforded pure dye (4 g, 32% yield), λmax=747 nm in methanol, extinction coefficient=220,020.

### EXAMPLE 2

### Dye Synthesis for Preparation of Dye 5

This dye was prepared following the general procedure described above, using 2,3,3-trimethyl-1-butyl-3H-Indolium perchlorate (12 g, 38 mmoles) and the dianil (5.4 g, 19 moles) in 100 mL of acetic anhydride containing tributylamine (10.5 g, 57 mmoles). The reaction was carried out for 15 minutes, cooled to 25 degrees and poured into 2000 mL of ice water with vigorous stirring. The water was decanted from the oily product then chromatographed on silica gel eluting with 90/10 methylene chloride/methanol. Evaporation of the solvent after drying with anhydrous magnesium sulfate afforded pure dye (8 g, 71% yield). λmax=746 nm in methanol with extinction coefficient of 259,500.

### EXAMPLE 3

### Dye Synthesis for Preparation of Dye 8

This dye was prepared following the general procedure described above using 1,2,3,3-tetramethyl-3H-Indolium borontetrabromide (5.22 g, 20 mmol) and the dianil (2.84 g, 10 mmol) in 25 mL of isopropyl alcohol containing acetic anhydride (3 ml) and triethylamine (5.6 ml) for 2 hours. The reaction was cooled to 25° C. and poured into 1 liter of ice water with vigorous stirring. The crude product was collected by filtration and washed again with water. The crude product was purified by recrystallization from hot ethyl alcohol. 3.4 g pure product was obtained. The ¹H NMR spectrum is consistent with the structure. λmax=739 nm in methanol, extinction coefficient=294,000

### EXAMPLE 4

### Dye Synthesis for Preparation of Dye 10

This dye was prepared following the general procedure described above, using 2,3,3-trimethyl-1-(4-sulfobutyl)-3H-Indolium inner salt (2.3 g, 6.7 mmoles) and the dianil (0.95 g, 3.3 moles) in 20 mL of acetic anhydride. Triethylamine (2 g, 20 mmoles) was added with vigorous stirring and the reaction heated to reflux for 5 minutes. The reaction was cooled and diluted to 300 mL with diethyl ether and stirred for 10 minutes. The ether was decanted from the oil and 25 mL of absolute ethanol was added. The mixture was heated to reflux then 1.5 g (0.01 moles) of sodium iodide was added. Heating was continued for 3 minutes and the mixture was cooled to 25 degrees with stirring. The solid was filtered, washed with absolute ethanol and dried. Wt=2.4 g (94% yield, 85% purity). The product was purified by reverse phase HPLC to yield 1 g of desired dye (39% yield, 99% purity by HPLC. λmax=784 nm methanol, extinction coefficient=221,700.

### EXAMPLE 5

### Dye Synthesis for Preparation of Dye 2

Dye 2 was synthesized using the synthetic scheme described in the literature (Weili Zhao and Erick M. Carreira Angew. Chem. Int. Ed. 2005, 44, 1677). λmax=739 nm in methanol, extinction coefficient=129945.

### EXAMPLE 6

### Dye Synthesis for Preparation of Dye 3

Isostearyl alcohol (1.8 g, 6.6 mmol) was mixed with N,N-dimethylformamide (50 ml), treated with sodium hydride (0.31 g of 50% oil mixture, 6.6 mmol) and stirred at ambient conditions under nitrogen atmosphere for 2 hrs. Silicon phthalocyanine dichloride was added and the reaction was heated at reflux overnight. The reaction was portioned between water and ethyl acetate and the organic layer was washed 3 times with water. The organic layer was dried over magnesium sulfate, filtered and concentrated. The residue was chromatographed on silica to yield 0.8 g of product. The ¹H NMR spectrum was consistent with the structure. λmax in toluene (672 nm)

### EXAMPLE 7

### Dye Synthesis for Preparation of Dye 4

A mixture of 3H-Indolium, 2,3,3-trimethyl-1-octadecyl-, perchlorate salt (2.14 g, 5 mmol), N-((2-chloro-3-((phenylamino)methylene)-1-cyclohexen-1-yl)methylene)-benzenamine monohydrochloride (0.9 g, 2.5 mmol) and 1-methyl-2-pyrrolidinone (30 ml) was heated at 60° C. overnight. The mixture was cooled to room temperature and poured into water. The dye was collected by filtration and washed with water and dried in a vacuum oven to afford the crude product (1.8 g). The crude dye was used in the next step without further purification.

To a solution of phenol (130 mg, 1.4 mmol) in anhydrous THF (20 ml) was added sodium hydride (60 mg, 1.4 mmol, 60% in mineral oil) at room temperature. The mixture was stirred for 30 minutes, then the dye (0.8 g, 0.9 mmol) was added and the mixture was heated at 60° C. for overnight. The solvent was then removed and residue was purified chromatographically (silica-gel column; dichloromethane with 2% Methanol) to give the pure dye product (560 mg). λmax=771 nm in acetone. Both the mass spectrum and H¹NMR are consistent with the structure.

### EXAMPLE 8

### Dye Synthesis for Preparation of Dye 6

To a round bottom flask charged with 3H-Indolium, 2,3,3-trimethyl-1-octadecyl-, p-toluenesulfonate (PTS) salt ( 5.75 g, 12 mmoles), N-(3-(phenylamino)-2-propenylidene)-benzenamine, monohydrochloride (1.55 g, 6 mmoles), and acetonitrile (15 ml) was added acetic anhydride (1.3 ml) and triethylamine (3.4 ml). The mixture was heated to reflux and a second portion of triethylamine (2.0 ml) was added. The resulting mixture was refluxed for two hours. After the mixture was cooled to room temperature, water was added while stirring. The crude dye with PTS as a counter ion (6.2 g) was collected by filtration and air-dried.

The PTS counter ion of the dye was next exchanged to perchlorate. To a solution of the dye (6.1 g) in methanol (55 ml) was added a solution of sodium perchlorate (1.3 g) in methanol. The mixture was stirred at room temperature for 1 hour and the dye was precipitated out and collected by filtration. The dye was further purified by recrystallization from methanol. 3.2 g of dye 6 was obtained, λmax=682 nm in methanol, extinction coefficient=2.33×10⁵.

### EXAMPLE 9

### Dye Synthesis for Preparation of Dye 7

This dye was prepared following the procedure of Dye Synthesis Example 7 with an additional ion exchange step. The condensation step used 3H-Indolium, 2,3,3-trimethyl-1-butyl, p-toluenesulfonate salt (10.76 g, 20 mmoles), N-(3-(phenylamino)-2-propenylidene)-benzenamine, monohydrochloride (2.6 g, 10 mmoles), acetonitrile (30 ml), acetic anhydride (2.5 ml) and triethylamine (6.5 ml). 10.2 gram of crude dye was obtained, the dye was ion exchanged to the perchlorate as described in Dye Synthesis example 7 using the crude dye (10 g, 18.6 mmoles), MeOH (70 ml), and sodium perchlorate (2.6 g, 21.2 mmoles). 8.2 g of the perchlorate dye was obtained. The crude perchlorate dye (5 g, 9.3 mmoles) was stirred in methanol (300 ml), with amberlite IRA-400 (Cl) resins (40 g) for several hours. The resin was filtered off and this was repeated with a second portion of resin. The solvent was removed on a rotary evaporator and the dye was dried overnight in a vacuum oven at 60° C. The final dye 7 was obtained (4.7 g) with λmax=641 nm in methanol, extinction coefficient=2.56×10⁵.

### EXAMPLE 10

### Dye Synthesis for Preparation of Dye 11

Dye 11 was prepared according to the procedures described by Lou Kai-yan, Qian Xu-huong, Song Gong-hua, Journal of East China University of Science and Technology, 28, (2), 212-5, 2002. λmax=751 nm in methanol. extinction coefficient=2.31×10⁵.

### EXAMPLE 11

### Preparation of benzoic acid functionalized polyethylene glycol methacrylate(PEG-MA) macromonomer

Synthesis of 4-Fuoro-3-nitrobenzoyl chloride: The 4-fluoro-3-nitrobenzoic acid (18.5 g, 0.1 mol MW 185.1) was mixed with 100 ml of thionyl chloride and heated at reflux with stirring for 3 hrs. The reaction was concentrated and dissolved in cyclohexane and reconcentrated.

The 4-Fuoro-3-nitrobenzoyl chloride was dissolved in methylene chloride 50 ml and added dropwise to a mixture of the polyethylene glycol methacrylate (52.6 g, Mw 526) methylence chloride (300 ml) and triethylene chloride (12 g) dropwise at room temperature. The reaction was stirred for 24hrs. The reaction product was concentrated and the residue was taken up in ethyl acetate. The organic layer was partitioned with saturated NaHCO₃ and dried and concentrated.

### EXAMPLE 12

### Preparation of Reactive Nanolatex 1 comprised of methoxyethyl methacrylate (55% w/w), divinylbenzene (6%), methoxy-poly(ethylene glycol) methacrylate (Mw:1100) (23%), and 4-fluoro-2-nitrobenzoyl-poly(ethylene glycol)methacrylate (Mw:687) (11%)

A 500 ml 3-neck round flask (referred as the “header” flask) was modified with Ace #15 glass threads at the bottom and a series of adapters were used to connect the bottom of flask with 1/16 inch ID Teflon tubing. The header flask was fitted with a mechanical stirrer, and rubber septum with syringe needle for nitrogen inlet. The header flask was charged with methoxyethyl methacrylate (6.88 g), divinylbenzene (0.63 g, mixture of isomers, 80% pure with remainder being ethylstyrene isomers), methoxy-poly(ethylene glycol)methacrylate (2.50 g, Mₙ=1100), 4-fluoro-2-nitrobenzoyl-poly(ethylene glycol)methacrylate (1.25 g, Mw:687), cetylpyridinium chloride (0.156 g), 2,2′-azobis(N,N′-dimethyleneisobutyramidine)dihydrochloride (0.06 g), and distilled water (78.38 g). A 1 L 3-neck round bottomed flask outfitted with a mechanical stirrer, reflux condenser, nitrogen inlet, and rubber septum (hereafter referred to as the “reactor”) was filled with 2,2′-azobis(N,N′-dimethyleneisobutyramidine) dihydrochloride (0.06 g), cetylpyridinium chloride (0.469 g), and distilled water (159.13 g). Both the header and reactor mixtures were stirred until homogeneous and were bubble degassed with nitrogen for 20 minutes. The reactor flask was placed in a water bath at 60° C. and the header monomers mixture were added to the reactor over two hours using a model QG6 lab pump (Fluid Metering Inc. Syossett, N.Y.). The reaction mixture was then allowed to stir at 60° C. for 20 hours. The reaction mixture was then dialyzed for 48 hours using a 3.5K cutoff membrane in a bath with continual water replenishment. Then, the reaction product (274.37 g) was treated by 30.59 g Dowex 50 W×4 ion exchange resin (converted to the sodium form and washed 3× with distilled water) for overnight under stirring. At last, the reaction products were filtrated to give nanolatex solution with 3.3% solids. The volume mean diameter and Zeta potential of this nanolatex particles were measured by Malvern Instrument (Model:ZEN3600) and found to be 37.6 nm, and −2.14 mV, respectively.

### EXAMPLE 13

### Preparation of Reactive Nanolatex 2 comprised of methoxyethyl methacrylate (45% w/w), divinylbenzene (5%), methoxy-poly(ethylene glycol) methacrylate (Mw:1100) (40%), and 4-fluoro-2-nitrobenzoyl-poly(ethylene glycol) methacrylate (Mw:687) (10%)

This nanolatex was prepared using the same method as described in Example 12. The header contained methoxyethyl methacrylate (5.63 g), divinylbenzene (0.63 g, mixture of isomers, 80% pure with remainder being ethylstyrene isomers), methoxy-poly(ethylene glycol)methacrylate (5 g, Mₙ=1100), 4-fluoro-2-nitrobenzoyl-poly(ethylene glycol)methacrylate (1.25 g, Mw:687), cetylpyridinium chloride (0.156 g), 2,2′-azobis(N,N′-dimethyleneisobutyramidine)dihydrochloride (0.06 g), and distilled water (78.38 g). The reactor contents were composed of distilled water (159.13 g), 2,2′-azobis(N,N′-dimethyleneisobutyramidine)dihydrochloride (0.063 g), and cetylpyridinium chloride (0.469 g). A clear dispersion of product was obtained. 250 g of this nanolatex was dialyzed for 48 hours using a 3.5K cutoff membrane and followed by treatment with 30 g Dowex 50 W×4 ion exchange resin (sodium form) to afford of an ion exchanged dispersion of 4.0% solids. The volume mean diameter of this nanolatex characterized by Malvern Instrument is 19.2 nm

### EXAMPLE 14

### Preparation of Reactive Nanolatex 3 with the Same Composition of Nanolatex 2 by Using Potassium Persulfate as Initiator Instead of Azo-Compound

This nanolatex was prepared using the same method as described in Example 12. The header contained methoxyyethyl methacrylate (5.63 g), divinylbenzene (0.63 g, mixture of isomers, 80% pure with remainder being ethylstyrene isomers), methoxy-poly(ethylene glycol)methacrylate (5.00 g, Mₙ=1100), 4-fluoro-2-nitrobenzoyl-poly(ethylene glycol)methacrylate (1.25 g, Mw:687), potassium persulfate (0.13 g), sodium bicarbonate (0.063 g), Dowfax 2A1 (1.736 g) and distilled water (78.38 g). The reactor contents were composed of distilled water (159.13 g), sodium metabisulfite (0.107 g), sodium bicarbonate (0.063 g), and Dowfax 2A1 (1.042 g). The latex was subjected to dialysis with a 3.5K cutoff membrane for 3 days. The latex was filtrated by 0.2 μm membrane and gave almost clear latex solution at solid percentage of 2.17. The volume mean diameter of the nanolatex particles was measured to be 39.2 nm by Malvern Instrument.

### EXAMPLE 15

### Preparation of Reactive Nanolatex 4 comprised of methoxyethyl methacrylate (60% w/w), divinylbenzene (5%), methoxy-poly(ethylene glycol) methacrylate (Mw:1100) (30%), and 4-fluoro-2-nitrobenzoyl-poly(ethylene glycol)methacrylate (Mw:687) (5%) by using another azo-compound as initiator

This nanolatex was prepared using the same method as described in Example 12. The header contained methoxyyethyl methacrylate (7.5 g), divinylbenzene (0.63 g, mixture of isomers, 80% pure with remainder being ethylstyrene isomers), methoxy-poly(ethylene glycol)methacrylate (3.75 g, Mₙ=1100), 4-fluoro-2-nitrobenzoyl-poly(ethylene glycol)methacrylate (0.63 g, Mw:687), 2,2′-azobis-{2-[1-(2-hydroxyethyl)-2-imidazolin-2-yl]propane}dichloride (0.08 g), cetylpyridinium chloride (0.156 g), and distilled water (78.38 g). The reactor contents were composed of distilled water (159.13 g), 2,2′-azobis-{2-[1-(2-hydroxyethyl)-2-imidazolin-2-yl]propane}dichloride (0.079 g), cetylpyridinium chloride (0.469 g). After dialysis, ion exchange resin treatment, a clear dispersion (211 g) with 4.12% solids was obtained. The volume mean diameter was found to be 27.7 nm and its Zeta potential is −1.47 mV by Malvern Instrument.

### EXAMPLE 17

### Preparation of Reactive Nanolatex 5 comprised of methoxyethyl methacrylate (50% w/w), divinylbenzene (5%), methoxy-poly(ethylene glycol) methacrylate (Mw:1100) (30%), and 4-fluoro-2-nitrobenzoyl-poly(ethylene glycol)methacrylate (Mw:687) (15%) by using another azo-compound as initiator

This nanolatex was prepared using the same method as described in Example 12. The header contained methoxyyethyl methacrylate (6.25 g), divinylbenzene (0.63 g, mixture of isomers, 80% pure with remainder being ethylstyrene isomers), methoxy-poly(ethylene glycol)methacrylate (3.75 g, Mₙ=1100), 4-fluoro-2-nitrobenzoyl-poly(ethylene glycol)methacrylate (1.88 g, Mw:687), 2,2′-azobis-{2-[1-(2-hydroxyethyl)-2-imidazolin-2-yl]propane}dichloride (0.08 g), cetylpyridinium chloride (0.156 g), and distilled water (78.38 g). The reactor contents were composed of distilled water (159.13 g), 2,2′-azobis-{2-[1-(2-hydroxyethyl)-2-imidazolin-2-yl]propane}dichloride (0.079 g), cetylpyridinium chloride (0.469 g). After dialysis and ion exchange resin treatment, an almost clear dispersion (248 g) with 2.79% solids was obtained. The volume mean diameter was found to be 51.7 nm and its Zeta potential is −2.38 mV by Malvem Instrument.

### EXAMPLE 7

### Preparation of Reactive Nanolatex 6 comprised of methoxyethyl methacrylate (45% w/w), divinylbenzene (5%), methoxy-poly(ethylene glycol) methacrylate (Mw:1100) (40%), and 4-fluoro-3-nitrobenzoyl-poly(ethylene glycol)methacrylate (Mw:687, the second benzoyl-PEG MA monomer) (10%)

This nanolatex was prepared using the same method as described in Example 12. The header contained methoxyyethyl methacrylate (5.63 g), divinylbenzene (0.63 g, mixture of isomers, 80% pure with remainder being ethylstyrene isomers), methoxy-poly(ethylene glycol)methacrylate (5.00 g, Mₙ=1100), 4-fluoro-3-nitrobenzoyl-poly(ethylene glycol)methacrylate (1.25 g, Mw:687), 2,2′-azobis-{2-[1-(2-hydroxyethyl)-2-imidazolin-2-yl]propane}dichloride (0.08 g), cetylpyridinium chloride (0.156 g), and distilled water (78.38 g). The reactor contents were composed of distilled water (159.13 g), 2,2′-azobis-{2-[1-(2-hydroxyethyl)-2-imidazolin-2-yl]propane}dichloride (0.079 g), cetylpyridinium chloride (0.469 g). The nanolatex product was dialyzed by 3.5 k cutoff membrane at room temperature for 48 h, followed by treatment of Dowex 50 W×4 ion exchange resin (sodium form) for overnight, and afforded 230 g clear dispersion solution with solid (3.45%). The volume mean diameter was found to be 16.9 nm and its Zeta potential is −3.33 mV by Malvern Instrument.

### EXAMPLE 18

### Preparation of Reactive Nanolatex 7 comprised of methoxyethyl methacrylate (50% w/w), divinylbenzene (5%), methoxy-poly(ethylene glycol) methacrylate (Mw:2100) (40%), and 4-fluoro-3-nitrobenzoyl-poly(ethylene glycol)methacrylate (Mw:687, the second benzoyl-PEG MA monomer) (5%)

This nanolatex was prepared using the same method as described in Example 12. The header flask contained methoxyyethyl methacrylate (6.25 g), divinylbenzene (0.63 g, mixture of isomers, 80% pure with remainder being ethylstyrene isomers), 50 wt % methoxy-poly(ethylene glycol)methacrylate water solution (5.00 g, Mₙ=2100), 4-fluoro-3-nitrobenzoyl-poly(ethylene glycol) methacrylate (0.63 g, Mw:687), 2,2′-azobis-{2-[1-(2-hydroxyethyl)-2-imidazolin-2-yl]propane}dichloride (0.08 g), cetylpyridinium chloride (0.156 g), and distilled water (73.38 g). The reactor flask was filled by distilled water (159.13 g), 2,2′-azobis-{2-[1-(2-hydroxyethyl)-2-imidazolin-2-yl]propane}dichloride (0.079 g), cetylpyridinium chloride (0.469 g). The nanolatex dispersion was dialyzed for 48 h by 3.5 k cutoff membrane. The clear nanolatex dispersion (239 g) with solid (3.36%) was obtained. The volume mean diameter was characterized as 19.4 nm and its Zeta potential is −13.2 mV by Malvern Instrument.

### EXAMPLE 19

### Preparation of Nanolatex 8 comprised of methoxyethyl methacrylate (50% w/w), divinylbenzene (5%), poly(ethylene glycol) methacrylate (average Mn:526) (40%), and 4-fluoro-3-nitrobenzoyl-hydroxyl-poly(ethylene glycol)methacrylate (Mw:687, the second benzoyl-PEG MA monomer) (5%)

This nanolatex was prepared using the same method as described in Example 12. The header flask contained methoxyyethyl methacrylate (6.25 g), divinylbenzene (0.63 g, mixture of isomers, 80% pure with remainder being ethylstyrene isomers), hydroxyl-poly(ethylene glycol)methacrylate (5.00 g, Mₙ=526), 4-fluoro-3-nitrobenzoyl-poly(ethylene glycol)methacrylate (0.63 g, Mw:687), 2,2′-azobis-{2-[1-(2-hydroxyethyl)-2-imidazolin-2-yl]propane}dichloride (0.08 g), cetylpyridinium chloride (0.156 g), sodium bicarbonate (0.13 g) and distilled water (78.38 g). The reactor flask was filled by distilled water (159.13 g), 2,2′-azobis-{2-[1-(2-hydroxyethyl)-2-imidazolin-2-yl]propane}dichloride (0.079 g), cetylpyridinium chloride (0.469 g). The nanolatex dispersion was dialyzed for 48 h by 3.5 k cutoff membrane and then treated by Dowex 50 W×4 ion exchange resin (sodium form) for overnight to give 219 g slightly translucent solution with solid (3.44%). The volume mean diameter was characterized as 34.1 nm and its Zeta potential is −5.91 mV by Malvern Instrument.

### EXAMPLE 20

### Loading of the Reactive Nanolatex 1 with Dye 1

Under dim light, a dye stock solution of 0.051 % w/w was prepared by dissolving 0.0153 g of Dye 1 in sufficient tetrahydrofuran (THF) to give a final solution at weight of 30.0095 g. 16.5780 g dye solution was added to a glass vial and was diluted to a final weight of 30.0 g with tetrahydrofuran. The nanolatex 1 (30.010 g) was added to the vial. THF solvent was evaporated from the mixture by rotary evaporator or by nitrogen gas stream. 20.95 g of a loaded reactive latex (LRL-1) of 4.97% solids containing 3.94×10⁻³ mol dye per gram of solid latex.

TABLE 1
Loading of the Reactive Latex 1 with Dye 1

| Loaded | | | | | |
|---|---|---|---|---|---|
| reactive | Dye | | | Conc. Dye in | Final % |
| latex | solution | Nanolatex | Final | solid latex | solids |
| designation | (g) | (g) | weight (g) | (mol/L) | (% w/w) |
| LRL-1 | 14.5780 | 30.0110 | 20.95 | 9.93 × 10⁻³ | 4.97 |

### EXAMPLE 21

### Loading of the Reactive Nanolatex 2 with Dye 1

Loaded LRL-2 nanolatex was prepared in brown glass vials from Nanolatex 2 using the procedure as described in Example 20 and the reagent quantities in the table below. In order to convert the latex serums to phosphate buffered saline, a salt mixture (137 parts NaCl, 2.7 parts KCl, 10 parts Na₂HPO₄, 2 parts KH₂PO₄) was added to the sample as listed below.

TABLE 2
Loading of Reactive Latex 2 with Dye 1

| Loaded | | | | | | |
|---|---|---|---|---|---|---|
| reactive | | | | | Conc. | Final |
| latex | Dye | | Final | Buffer | Dye in | % |
| desig- | solution | Nanolatex | weight | salts | solid latex | solids |
| nation | (g) | (g) | (g) | (g) | (mol/L) | (% w/w) |
| LRL-2 | 6.7440 | 30.2910 | 20.79 | 0.2083 | 9.83 × | 5.82 |
| | | | | | 10⁻³ | |

### EXAMPLE 22

### Loading of Reactive Nanolatex 4 with Dye 1 and Dye 7

Dye 1 loaded latex LRL-4A was prepared from Reactive Nanolatex 4 by using the procedure described in Example 20. Dye 1 loaded latex LRL-4B was prepared by sonication of dye and latex mixture. 3.4520 g Dye 1 THF solution (0.0981 wt % ) was added to a brown glass vial and diluted to 20 g. 10.0230 g nanolatex 4 aqueous dispersion was fitted to the vial with 20 g Dye 1 THF solution. The Dye 1 and nanolatex 4 mixture in water/THF miscible solution was sonicated for 10 min in water bath. THF was evaporated from the mixture solution by rotary evaporator. PBS salts were added to LL-4A.

Under dim light, a Dye 7 stock solution of 0.1307% w/w was prepared by dissolving 0.0335 g Dye 7 in THF to afford a final solution at weight of 25.034 g. 6.232 g dye solution was added to a brown glass vial and diluted to a final weight of 30.0 g with THF. The nanolatex 4 (31.1920 g) was filled in the vial. THF solvent was evaporated from the mixture by nitrogen gas stream to give loaded LL-4C. Similar with LL-4B, Dye 7 loaded LL-4D was prepared by sonication the mixture of Dye with latex, followed by evaporation of THF via rotary evaporator.

All reagents for LL-4A, 4B, 4C and 4D are shown in the table.

TABLE 3
Loading of Reactive Nanolatex 4 with Dye 1 and Dye 7

| Loaded | | | | | | |
|---|---|---|---|---|---|---|
| reactive | | | | | Conc. Dye in | Final % |
| latex | Dye | Nanolatex | Final | Buffer | solid latex | solids |
| designation | solution (g) | (g) | weight (g) | salts (g) | (mol/L) | (% w/w) |
| LRL-4A | 10.3920 | 30.0920 | 21.00 | 0.1997 | 9.89 × 10⁻³ | 5.84% |
| LRL-4B | 3.4520 | 10.0230 | 7.94 | No | 9.94 × 10⁻³ | 5.15% |
| LRL-4C | 6.2320 | 31.1920 | 23.24 | No | 9.62 × 10⁻³ | 5.54% |
| LRL-4D | 2.2060 | 10.0520 | 8.61 | No | 1.06 × 10⁻² | 4.84% |

### EXAMPLE 23

Loading of Reactive Nanolatex 5 with Dye 1 and 7

Dye 1 loaded latex LRL-5A, and Dye 7 loaded LL-5B were prepared from reactive Nanolatex 5 using the procedure described in Examples 20 and 22. The Dye1 stock solution (0.1049% w/w, 20.9631 g) and Dye 7 stock solution (1.1207% w/w, 25.634 g) were prepared by dissolving 0.0220 g Dye 1 and 0.0335 g Dye 7 in tetrahydrofuran. All reagents for LRL-5A and 5B are listed in below Table.

TABLE 4
Loading of Reactive Nanolatex 5 with Dye 1 and Dye 7

| Loaded | | | | | | |
|---|---|---|---|---|---|---|
| reactive | | | | Buffer | Conc. Dye in | Final % |
| latex | Dye | Nanolatex | Final | salts | solid latex | solids |
| designation | solution (g) | (g) | weight (g) | (g) | (mol/L) | (% w/w) |
| LRL-5A | 2.182 | 10.051 | 7.59 | 0.0615 | 9.91 × 10⁻³ | 3.54% |
| LRL-5B | 4.319 | 30.427 | 23.56 | No | 1.01 × 10⁻² | 3.59% |

### EXAMPLE 24

### Loading of Reactive Nanolatex 6, 7 and 8 with Dye 1

Loaded reactive latex LRL-6, 7 and 8 was prepared from Nanolatex 6, 7 and 8, respectively, using the procedure described in Example 20. The reagent quantities are given in the table below. The dye stock solution (0.0358% w/w) was prepared by dissolving 0.0212 g of Dye 1 in sufficient tetrahydrofuran to afford a final solution weight of 20.082 g.

TABLE 5
Loading of Reactive Nanolatex 6, 7 and 8 with Dye 1

| Loaded | | | | | | |
|---|---|---|---|---|---|---|
| reactive | | | | Buffer | Conc. Dye in | Final % |
| latex | Dye | Nanolatex | Final | salts | solid latex | solids |
| designation | solution (g) | (g) | weight (g) | (g) | (mol/L) | (% w/w) |
| LRL-6 | 2.7450 | 10.032 | 7.4619 | 0.0760 | 1.02 × 10⁻² | 4.12% |
| LRL-7 | 2.6120 | 10.024 | 6.3922 | 0.0651 | 9.93 × 10⁻³ | 4.54% |
| LRL-8 | 2.7850 | 10.145 | 6.9800 | 0.0685 | 1.02 × 10⁻² | 4.41% |

### EXAMPLE 25

### Conjugation of Reactive Nanolatex with Peptide and Antibody

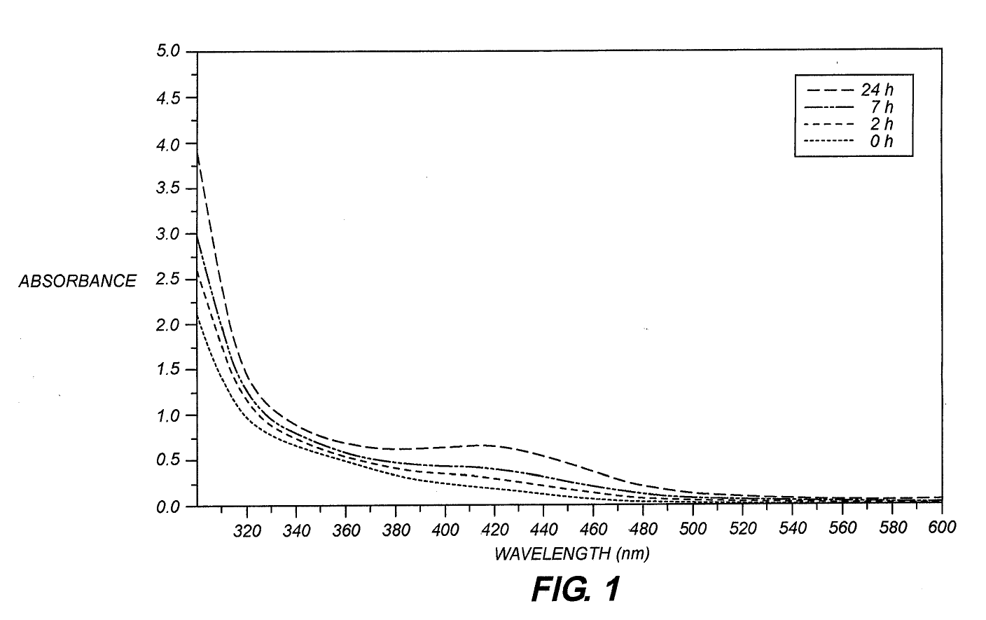

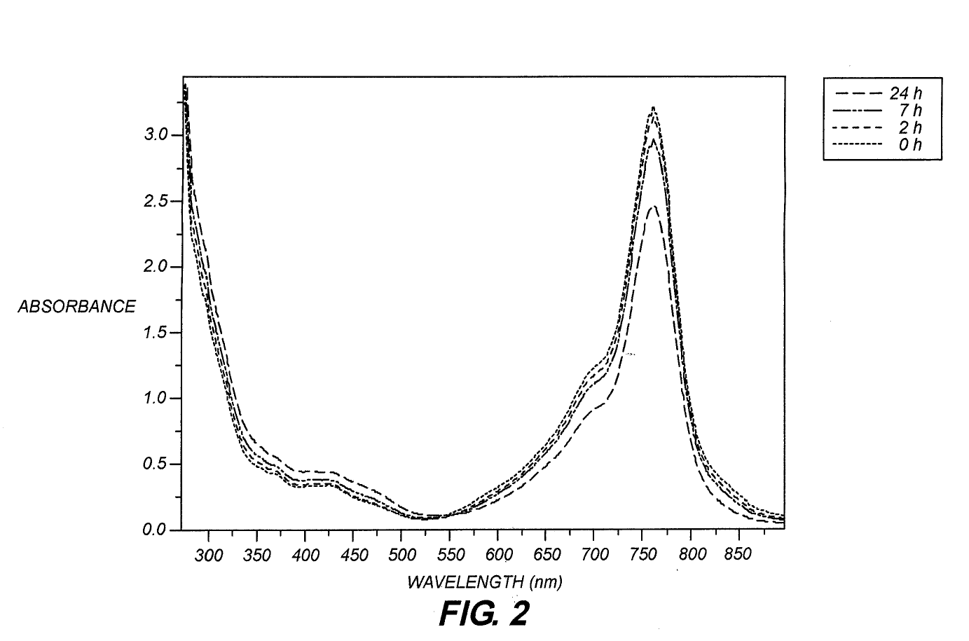

Fluoro-group attached to benzoyl-PEG-MA on surface of the reactive nanolatex particle can be used to directly conjugate with any primary or secondary amine containing peptide, bioactive ligands, proteins, antibody or drugs by formation of imino link —NH—. Lysine monohydrochloride (H₂N(CH₂)₄CH(NH₂)COOH.HCL, F.W. 182.65) with two —NH₂ group was employed to conjugate with reactive nanolatex. For an example, 7.61 mg lysine monohydrochloride was dissolved in 10 g reactive nanolatex (solid percentage: 0.43%, 20 wt % 4-fluoro-2-nitro-benzoyl-PEG-MA) in phosphate-buffered saline (PBS buffer, pH 7.4). The vial with latex and lysine in PBS buffer was placed in oil bath at 37° C. The nanolatex particle was conjugated with lysine at 37° C. for 2 h, 7 h and 24 h. Their UV-visible absorption spectra and the spectrum before conjugation (conjugation 0 h), which were measured by using a Perkin Elmer Lambda 900 UV/VIS/NIR spectrometer, are shown in FIG. 1. After conjugation at 37° C. for 24 h, a new peak at about 418 nm appeared, which indicates that the fluoro-group on benzoyl reacted with amine (—NH₂) in lysine and formed imino (—NH—) link with lysine. It has been reported (David L. Ladd and Robert A. Snow, “Reagents for the preparation of chromophorically labeled polyethylene glycol-protein conjugates”, Analytical Biochemistry, 1993, 210, 258-261.) that 4-fluoro-3-nitro-benzoyl-PEG was conjugated with ε-amine of lysine-containing proteins. The new absorption peak at 428 nm caused by nitroaniline after conjugation was used to characterize the conjugation degree. From FIG. 1, the absorption intensity for latex-lysine conjugate increases with an increase of conjugation time. This result demonstrates that the conjugation degree of reactive latex increases with conjugation time. Another example is the conjugation of Dye 1 loaded nanolatex LRL-4A (5 wt % 4-fluor-2-nitro-benzoyl-PEG-MA) with lysine (shown in FIG. 2).

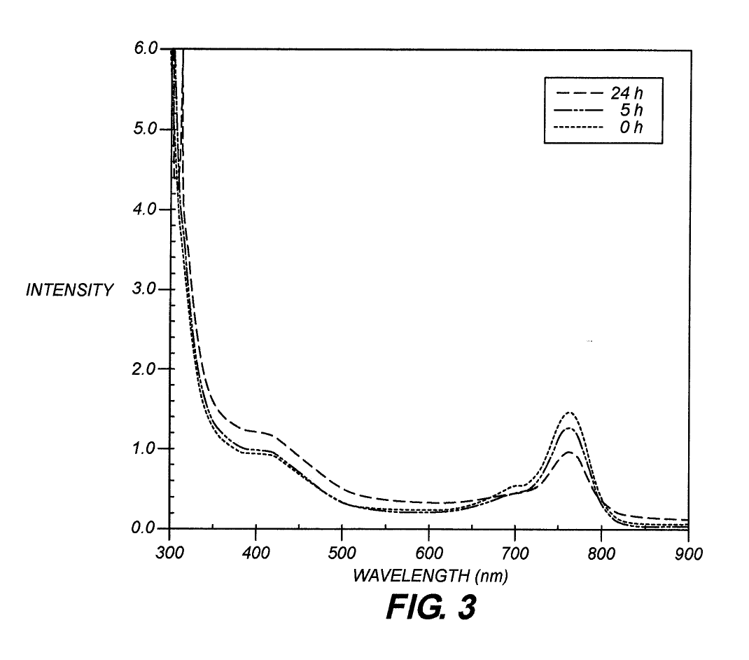

Dye 1 loaded nanolatex LRL-5A (15 wt % 4-fluor-2-nitro-benzoyl-PEG-MA) was conjugated with antibody. 1 mg anti-rabbit IgG antibody was dissolved in 0.500 g PBS buffer and formed slightly translucent solution. This IgG antibody solution was mixed with 0.500 g nanolatex LL-5A (0.8 wt % solid, 15 wt % 4-fluor-2-nitro-benzoyl-PEG-MA) and gave a mixture in PBS buffer with solid 0.4 wt %. The nanolatex and IgG mixture was placed in 37° C. oil bath for conjugation. The UV-visible spectra of this mixture before conjugation (0 h), and after conjugation 5 h and 24 h were measured and shown in FIG. 3. It is clear that the conjugation of latex with antibody can be carried within 5 h at 37° C. and the conjugation degree also increases with the conjugation time, which is similar with the conjugation between nanolatex and lysine. Sephacryl 500 column was employed to separate the IgG-latex conjugates from free IgG antibody.

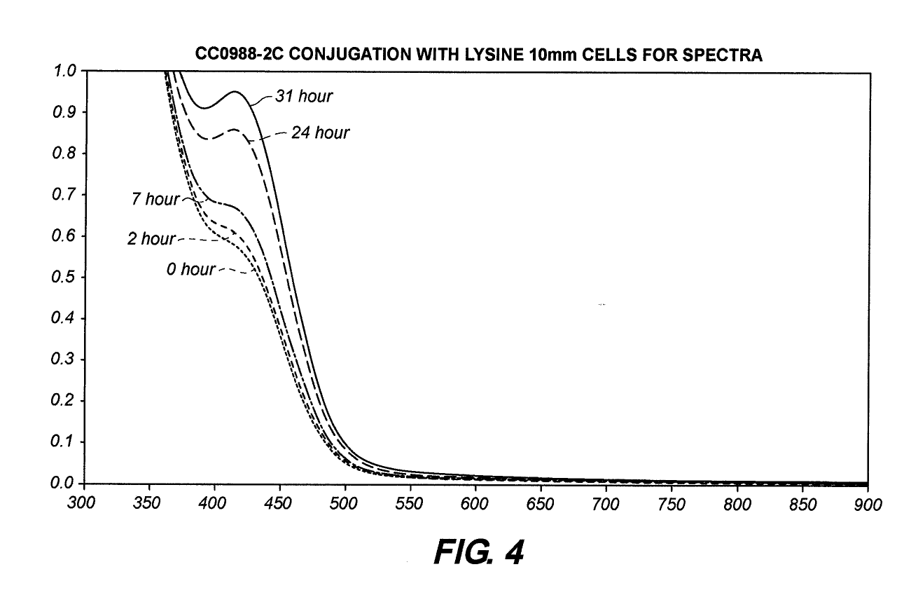

The reactive nanolatex containing 4-fluoro-3-nitro-benzoyl reactive groups was also used to conjugate with lysine at 37° C. for different time. Its UV-visible absorption spectra before conjugation (0 h) and after conjugation for 2 h, 7 h, 24 h and 31 h are shown in FIG. 4. Similar with the reactive nanolatex containing 4-fluoro-2-nitro-benzoyl reactive groups, the new absorption peak around 418 nm appeared after conjugation with lysine for 24 h and 31 h. The conjugation degree of the reactive latex with 4-fluoro-3-nitro-benzoyl reactive groups also increases with the conjugation time. At the same conjugation conditions, the conjugation degree of the reactive nanolatex containing 4-fluoro-3-nitro-benzoyl reactive groups is little bit higher than that of the latex with 4-fluoro-2-nitro-benzoyl reactive groups. The conjugation conditions, such as the concentration of reactive fluoro-nitro-benzoyl groups, ratios of reactive group/peptide, and conjugation time and temperature, have some effects on conjugation degree.

### EXAMPLE 26

### Fluorescence Relative Quantum Yield (RQY) Measurements

Aqueous dyed reactive nanolatex dispersions were diluted volumetrically with phosphate-buffered saline (PBS, pH 7.4) to a typical final dye concentration of 10⁻⁷ to 10⁻⁸ moles/L, such that their peak absorbance intensity at λ-max did not exceed 0.1. The standard (reference) dye sample was similarly prepared by dissolving the solid dye in spectroscopic-grade methanol at room temperature followed by further dilution with either PBS buffer or methanol to a dye concentration of 10⁻⁷ to 10⁻⁸ moles/L. The final dye-in-PBS solutions typically contained less than 1% methanol.

All dilutions were performed under dim light conditions and the samples were stored in amber borosilicated vials to minimize photodecomposition. All samples were measured within hours of dilution.

Absorption measurements were made by using a Perkin Elmer Lambda 900 UV/VIS/NIR spectrometer with dye solutions in 5 cm path length cuvettes. The absorption intensity of the dye was measured at the specific excitation wavelength used for the fluorescence measurement from the solvent-subtracted and baseline-zeroed absorption spectrum. These measured absorption values were then changed to 1 cm path length cuvette-equivalent values for the RQY calculations.

The fluorescence spectrum of the dye solution in a 1 cm path length cuvette was recorded in right-angle detection mode using a SPEX fluorolog 1680 0.22 m double spectrometer. The instrumental parameter settings and the excitation wavelength used for the inventive and reference dyes were co-optimized and were identical for each experiment. The baseline-resolved fluorescence spectrum of each dye sample was corrected for solvent contributions and instrumental response characteristics as a function of emission wavelength and the integrated fluorescence intensity was measured.

The integrated fluorescence intensity for each dye-containing sample was divided by the 1 cm pathlength-equivalent absorbance measured at the excitation wavelength of interest. The calculated F/A value is proportional to the dye's fluorescence quantum yield. The F/A value for the inventive dye was then normalized to the F/A value for the reference dye, multiplied by reference dye's known fluorescence quantum yield and corrected for any solvent refractive index differences— to yield a relative quantum yield (RQY) value for the inventive dye. The data are presented in Table 6.

TABLE 6
Absorption and fluorescence data of dye-loaded reactive nanolatex

| | | | | Relative quantum |
|---|---|---|---|---|
| | | λ-a (nm) | λ-e (nm) | yield |
| Latex samples | Dye | (absorption) | (emission) | Φ_{f} |
| LRL-1 | Dye 1 | 762 | 786 | 0.1 |
| LRL-2 | Dye 1 | 763 | 784 | 0.1 |
| LRL-4A | Dye 1 | 761 | 786 | 0.076 |
| LRL-4B | Dye 1 | 760 | 787 | 0.058 |
| LRL-4C | Dye 7 | 689 | 715 | 0.22 |
| LRL-4D | Dye 7 | 689 | 715 | 0.20 |
| LRL-5A | Dye 1 | 762 | 788 | 0.045 |
| LRL-5B | Dye 7 | 691 | 715 | 0.09 |
| LRL-6 | Dye 1 | 762 | 785 | 0.091 |
| LRL-7 | Dye 1 | 762 | 786 | 0.11 |
| LRL-8 | Dye 1 | 761 | 786 | 0.10 |

All Dye 1 loaded reactive nanolatex exhibited similar absorption wavelength (from 760 nm to 763 nm) and similar emission wavelength (784 nm-788 nm). Their fluorescence quantum yield values are from 0.045 to 0.11, which are much higher than that of the hydrophilic near-infrared Indocynanine Green (Dye 10 with RQY at about 0.006) in an aqueous solution. Dye 7 loaded reactive nanolatex also showed similar absorption at 689-691 nm and the same emission at about 715 nm. Some samples (LL-4C and 4D) have pretty high fluorescence quantum yield (≧0.20).

1. A loaded reactive nanolatex particle comprising:
a crosslinked latex polymer made from a mixture represented by formula:
(X)m-(Y)n-(V)q-(T)o-(W)p,
m, n, q, o, and p represent the weight percentages of each component;
X is a water-insoluble, alkoxyethyl-containing monomer represented by the formula: 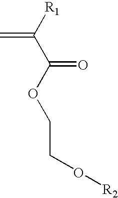
where R1 is hydrogen or methyl; R2 is an alkyl or aryl group containing up to 10 carbons,
Y is at least one monomer containing two ethylenically unsaturated chemical functionalities;
V is a polyethyleneglycol-methacrylate derivative represented by the formula: 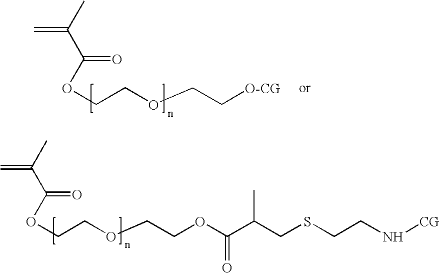
where n is greater than 1 and less than 130 and CG is selected from the group consisting of: 4-halo-3-nitrobenzoyl, 2-halo-3-nitrobenzoyl, 2-halo-4-nitrobenzoyl, 4-halo-2-nitrobenzoyl, 2-halo-5-nitrobenzoyl, 3-halo-2-nitrobenzoyl, 2-halonicotinate, 4-halonicotinate, 6-halonicotinate 2-haloisonicotinate, and 3-haloisonicotinate, where halo is selected from the group consisting of: fluoro, chloro, bromo, and iodo;
T is a polyethyleneglycolacrylate containing macromonomer represented by the formula: 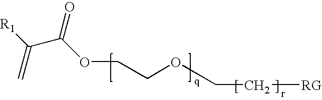
where R1 is hydrogen or methyl, q is 5-220, r is 1-10, and RG is a selected from the group consisting of: hydrogen, hydroxyls, carboxylic acids, vinylsulfonyls, aldehydes, epoxides, succinimidyl esters and maleimides;
W is an ethylenic monomer different from X, Y, V, or T;
wherein m ranges between 40-80 wt %,
n ranges between 1-10 wt %,
q ranges between 1-30 wt %,
o ranges between 20-50 wt %, and
p is less than 10 wt % ;
said particle is loaded with a molecular imaging agent.

2. The loaded reactive nanolatex particle of claim 1, wherein said loaded reactive nanolatex particle is biocompatible and has a hydrodynamic diameter of less than 100 nm.

3. The loaded reactive nanolatex particle of claim 1, further comprising a biological, pharmaceutical or diagnostic component associated with an outer surface of the nanolatex particle by chemically bonding the biological, pharmaceutical or diagnostic component with a reactive or functional group selected from the group consisting of: 4-fluoro-3-nitro-benzoyl, 4-fluor-2-nitro-benzoyl, hydroxyl, carboxylic acid, vinylsulfonyl, aldehyde, epoxide, succinimidyl ester and maleimide.

4. The loaded reactive nanolatex particle of claim 1, further comprising a targeting agent associated with the outer surface of the nanolatex particle by conjugation with functional groups on the nanolatex surface, wherein said targeting agent is capable of specifically binding with a biological site under physiological conditions.

5. The loaded reactive nanolatex particle of claim 1, wherein said molecular imaging agent is a fluorescent dye present in the loaded reactive nanolatex particle in an amount from 0.01 to 5 wt % , said fluorescent dye having a relative quantum yield of at least 0.01.

6. The loaded reactive nanolatex particle of claim 1, wherein said crosslinked latex polymer comprises:
at least 45 wt % water insoluble monomers; and
from 1 to 30 wt % monomers containing a reactive halo aromatic conjugating group.

7. The loaded reactive nanolatex particle of claim 1, wherein:
X is selected from the group consisting of: methoxylethyl, methacrylate and methoxylethyl acrylate;
V is a polyethyleneglycol-methacrylate derivative having a reactive fluoro-nitro-benzoyl group at the end of said polyethyleneglycol-methacrylate derivative and having an average molecular weight from 500 to 5,000;
T is a water soluble poly(ethylene glycol)methacrylate having an average molecular weight between 300 to 10,000.

8. The loaded reactive nanolatex particle of claim 1, wherein W is a water-soluble monomer selected from the group consisting of: 2-phosphatoethyl acrylate potassium salt, 3-phosphatopropyl methacrylate ammonium salt, vinylphosphonic acid, and their salts thereof, vinylcarbazole, vinylimidazole, vinylpyrrolidone, vinylpyridines, acrylamide, methacrylamide, maleic acid and salts thereof, sulfopropyl acrylate and methacrylate, acrylic and methacrylic acids and salts thereof, N-vinylpyrrolidone, acrylic and methacrylic esters of alkylphosphonates, acrylic and methacrylic monomers containing amine or ammonium, styrenesulfonic acid and salts thereof, acrylic and methacrylic esters of alkylsulfonates, vinylsulfonic acid and salts thereof, vinylpyridines, hydroxyethyl acrylate, glycerol acrylate and methacrylate esters, methacrylamide, and N-vinylpyrrolidone.

9. The loaded reactive nanolatex particle of claim 1, wherein W is a water-insoluble monomer selected from the group consisting of: methyl methacrylate, ethyl methacrylate, isobutyl methacrylate, 2-ethylhexyl methacrylate, benzyl methacrylate, cyclohexyl methacrylate, glycidyl methacrylate, acrylic/acrylate esters, styrenics, vinyl halides, vinylidene halides, N-alkylated acrylamides and methacrylamides, vinyl esters, vinyl ether, allyl alcohol ethers and esters, unsaturated ketones and aldehydes.

10. The loaded reactive nanolatex particle of claim 1 wherein T is a water soluble polyethyleneglycol (PEG) macromonomer having a functional group at the end of PEG with molecular weight from 500 to 5,000.

11. The loaded reactive nanolatex particle of claim 10 wherein said functional groups at the end of PEG is selected from the group consisting of: hydroxyl, carboxylic acid, vinylsulfonyl, aldehyde, epoxide, succinimidyl ester and maleimide.

12. The loaded reactive nanolatex particle of claim 10 wherein the functional group on latex surface is servable as an attachment point for a metal chelating group used to form a link between the loaded latex and a metal ion.

13. The loaded reactive nanolatex particle of claim 1 comprising at least one molecular imaging agent further comprising a second imaging agents selected from the group consisting of: position emission tomography agents, magnetic resonance imaging agents, radiological imaging agents and imaging agents for single photon emission computerized tomography, where said second imaging agent is physically incorporated into the reactive nanolatex particle.

14. The loaded reactive nanolatex particle of claim 1 comprising at least one molecular imaging agent further comprising a second imaging agents selected from the group consisting of: position emission tomography agents, magnetic resonance imaging agents, radiological imaging agents and imaging agents for single photon emission computerized tomography, where said second imaging agent is covalently bonded to a functional group on the surface of the nanolatex particle.

15. The loaded reactive nanolatex particle of claim 1 comprising at least one molecular imaging agent further comprising a second imaging agents selected from the group consisting of: position emission tomography agents, magnetic resonance imaging agents, radiological imaging agents and imaging agents for single photon emission computerized tomography, where said second imaging agent is associated with said nanolatex particle by a chelating group linked to the surface of the nanolatex particle.

16. A loaded reactive nanolatex particle comprising:
a crosslinked polymer represented by the formula:
(X)b-(Y)c-(V)d-(T)e-(W)f
X is a water-insoluble, alkoxyethyl-containing monomer represented by the formula: 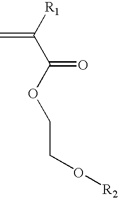
where R1 is hydrogen or methyl, and R2 is an alkyl or aryl group containing up to 10 carbons,
Y is at least one monomer containing two ethylenically unsaturated chemical functionalities;
V is a polyethyleneglycol-methacrylate derivative represented by the formula: 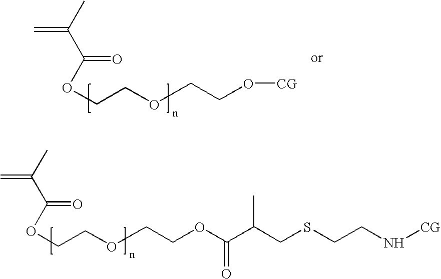
where n is greater than 1 and less than 130 and CG is selected from the group consisting of: aromatic sulfonates, alky sulfonates, electron withdrawing phenols and hetercyclic thiols;
T is a polyethyleneglycolacrylate containing macromonomer represented by the formula 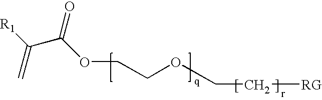
where R1 is hydrogen or methyl, q is 5-220, r is 1-10, and RG is a selected from the group consisting of: hydrogen, hydroxyls, carboxylic acids, vinylsulfonyls, aldehydes, epoxides, succinimidyl esters and maleimides
W is an ethylenic monomer different from X, Y, V, or T;
wherein b ranges from 40 to 80 wt %,
c ranges from 1 to 10 wt %,
d ranges from 1 to 30 wt %,
e ranges from 10 to 60 wt %, and
f is less than 10 wt %;
said crosslinked polymer comprising:
at least 45 wt % water insoluble monomers;
from 1 to 30 wt % monomers containing a reactive halo-aromatic conjugating group; and
a molecular imaging agent loaded within said reactive nanolatex particle.

17. The loaded reactive nanolatex particle of claim 16 wherein:
b ranges from 45 to 60 wt %,
c ranges from 2 to 6 wt %,
d ranges from 5 to 20 wt %, and
e ranges from 20 to 50 wt %.

18. The loaded reactive nanolatex particle of claim 16, wherein:
X is methoxylethyl methacrylate; and
V is fluoro-nitro-benzoyl-polyethyleneglycol-methacrylate having an average molecular weight from 500 to 5,000.

19. The loaded reactive nanolatex particle of claim 16, wherein said crosslinked polymer is PEGylated and has a volume average hydrodynamic diameter of less than 100 nm.

20. A method for forming a loaded reactive latex particle comprising the steps of:
synthesizing a mixture of a plurality of monomers comprising;
a first monomer having at least two ethylenically unsaturated chemical functionalities having a concentration between 1-10 wt %;
a second monomer, having a concentration between 1-30 wt %, being a polyethyleneglycol-methacrylate derivatives with a reactive fluoro-nitro-benzoyl group at the end of said polyethyleneglycol-methacrylate derivative and having an average molecular weight from 500 to 5,000;
a third monomer, having a concentration between 1-30 wt % being a water soluble poly(ethylene glycol)methacrylate with or without end functional groups having an average molecular weight between 300 to 10,000;
a fourth monomer is selected from the group consisting of:
methoxylethyl methacrylate and methoxylethyl acrylate, having a concentration between 20-50 wt %; and
a fifth monomer is an ethylenic monomer different from the first, second, third and fourth monomers having a concentration less than 10 wt %; in order to form a particle; and
loading said particle with a molecular imaging agent.
